(19) United States
(12) Patent Application Publication
(10) Pub. No.: US 2025/0090568 A1
(43) Pub. Date: Mar. 20, 2025

(54) METHOD OF TREATING LONG-COVID INDUCED NEUROLOGIC DISEASES

(71) Applicant: University of South Florida, Tampa, FL (US)

(72) Inventors: Subhra Mohapatra, Lutz, FL (US); Shyam S. Mohapatra, Lutz, FL (US); Paula C. Bickford, Ruskin, FL (US); Laura Janelle Blair, Riverview, FL (US); Bala Chandran, Tamp, FL (US); Karthick Mayilsamy, Tampa, FL (US); Ryan Green, New Port Richey, FL (US)

(21) Appl. No.: 18/959,846

(22) Filed: Nov. 26, 2024

Related U.S. Application Data
(63) Continuation of application No. PCT/US2023/026607, filed on Jun. 29, 2023.
(60) Provisional application No. 63/367,233, filed on Jun. 29, 2022.

(51) Int. Cl. A61K 31/7105; A61K 9/00; A61P 25/28; C12Q 1/6809

(52) U.S. Cl. CPC A61K 31/7105; A61K 9/0043; A61P 25/28; C12Q 1/6809; C12Q 2600/158

(57) ABSTRACT

A method of treating or preventing Alzheimer's disease or related dementias in patients previously infected with a respiratory virus such as SARS CoV2 is presented. Brain gene expression profiles of severe COVID-19 patients show increased expression of several innate immune response genes and genes implicated in Alzheimer's disease pathogenesis. The gene expression signature includes genes involved in inflammation, protein folding/trafficking, complement activation, calcium homeostasis, and amyloid/tau processing. The gene expression signature is correlated with tau pathology, α-synuclein, and demyelination with neuroinflammation being increased in old versus young CoV-2 infected mice.

### CROSS REFERENCE TO RELATED APPLICATIONS

This application is a continuation of and claims priority to International Application Serial No. PCT/US2023/026607, entitled “Method of Treating Long-COVID Induced Neurologic Diseases”, filed Jun. 29, 2023 which claims priority to U.S. Provisional Patent Application Ser. No. 63/367,233, entitled “Method of Treating Long-COVID Induced Neurologic Diseases”, filed Jun. 29, 2022, the contents of which are hereby incorporated by reference into this disclosure.

### GOVERNMENT SUPPORT

This invention was made with Government support under Grant No. BX005490 and BX005757 awarded by the Department of Veterans Affairs. The Government has certain rights in the invention.

### FIELD OF INVENTION

This invention relates to treatment and/or prevention of neurologic diseases. Specifically, the invention provides a method of preventing and/or treating neurologic diseases such as Alzheimer's disease and related dementias in patients previously infected with a respiratory virus such as SARS CoV2.

### BACKGROUND OF THE INVENTION

Alzheimer's disease and related dementias (ADRDs) are the most crippling cognitive threat to our aging population. By 2050, it is expected that the United States will spend $1.2 trillion to maintain the constantly deteriorating quality of life of 16 million Americans with AD,¹ including 5.5 million Americans ages 65 and older. There is no effective treatment or cure for ADRDs, and this is partly due to poor understanding of the underlying mechanisms and the diverse risk factors. Clinically, AD manifests as progressive cognitive decline and worsening memory deficits,² which have been critically linked to the aberrant accumulation of tau protein in neurons.³⁻⁵

“Inflammaging” is another common hallmark of ADRD, which can be exacerbated by the state of chronic inflammation triggered by pathological microbes, including viruses and their toxic metabolites. Previously, the “infectious AD hypothesis” was proposed based on the reactivation of neurotropic viruses such as herpes simplex virus (HSV), cytomegalovirus (CMV), and human herpes virus (HHV) 6, which caused the deposition of protein aggregates leading to cognition impairment.⁶ Also, the 1918 influenza outbreak reportedly resulted in a significant increase in Parkin-son's disease cases in years following.⁷ Epidemiological and laboratory rodent studies show the potential for respiratory viruses to have a neurological impact, sometimes in the absence of direct viral infection in the central nervous system (CNS).⁸ Although the precise mechanisms are poorly understood, it has been suggested that infections induce innate immune activation, causing neuroinflammation, which, in turn, promotes tau pathogenesis.⁹⁻¹² Microglia can become activated by tau oligomers, thus promoting a feed-forward cycle of inflammation and neurotoxicity.¹³ Once a positive regulatory loop between tau production and inflammation is established, the progression to ADRDs is no longer dependent on the initial cause of inflammation.

Severe acute respiratory syndrome coronavirus 2 (SARS-CoV-2) has caused the coronavirus disease 19 (COVID-19) pandemic with ˜600 million confirmed infections thus far¹⁴ and with long-term sequelae (long COVID) presenting in a significant proportion of infected individuals.¹⁵ A subgroup of severe long-COVID cases show a major neurological component with evidence of neuroinvasion correlating with death.¹⁶ About 80% of CoV-2-infected patients show CNS manifestations including dizziness, headache, loss of smell/taste, impaired consciousness, ataxia, epilepsy, encephalitis, and acute cerebrovascular disease, which are 40% more common in severe COVID-19 cases than in mild disease.¹⁷⁻²⁰ COVID-19 is also associated with ischemic stroke,^{21,22} encephalopathy,^{19,23}, syncope,²⁴ fluid-attenuated inversion recovery (FLAIR) hyperintensities,²⁵ and brain stem infections.²⁶ A comparison of magnetic resonance images (MRIs) in 51- and 81-year-old subjects before and after CoV-2 infections revealed significantly decreased orbitofrontal cortex thickness.²⁷ Moderate and severe COVID-19 have been associated with changes in brain structure and blood flow.^{27,28} Despite progress made, there is a paucity of knowledge regarding the cellular and molecular mechanisms by which the CoV-2 infection impacts the brain. There is an urgent unmet need to identify whether these changes lead to ADRD so that effective therapeutic interventions can be developed to ameliorate risk for ADRD following CoV-2 infection.

Previously, bioinformatic analyses of gene expression changes in COVID-19 patients identified significant immune dysregulation involving complement activation.²⁹ Also, CoV-2 contains 16 non-structural proteins and 8 open reading frame accessory genes, of which NSP1, ORF3a/3b, ORF6, ORF7a/7b, ORF8, and ORF9b are known to dysregulate the anti-viral response, creating the immunopathology observed in COVID-19.^{30,31}

Given the lack of available treatments for ADRD, what is needed is a method of assessing risk and correspondingly treating and/or preventing risk of development of neurologic disorders such as ADRD in patients previously infected with a respiratory virus such as SARS CoV-2.

### SUMMARY OF INVENTION

The coronavirus disease 2019 (COVID-19) pandemic has caused over 600,000,000 infections globally thus far. Up to 30% of individuals with mild to severe disease develop long COVID, exhibiting diverse neurologic symptoms including dementias. However, there is a paucity of knowledge of molecular brain markers and whether these can precipitate the onset of Alzheimer's disease (AD).

The inventors examined innate immune changes related to dysregulation of the immune anti-viral response to determine if it play key roles in ADRD onset and/or progression. The inventors analyzed gene expression in the brains of deceased CoV-2 patients and uninfected patients (NCBI Gene Expression Omnibus [GEO] database Series GEO: GSE188847) and compared these data with AD brains versus controls (GEO: GSE118553). These analyses identified several “hub” genes that are similarly regulated in both diseases and led us to hypothesize that CoV-2 can become neurotropic in aged brains causing innate immune activation, neuroinflammation, and neurodegeneration leading to tauopathy and the cognition impairment of AD. The inventors established CoV-2 infection in a mouse model using a mouse-adapted strain of SARS-CoV-2 (MA10) and examined how susceptibility to this viral infection changes with age. They further examined the association of gradual aging with inflammation, neurodegeneration, and infection-driven expression of ADRD genes/proteins with potential to cause the onset and/or rapid progression of ADRD.

The inventors discovered a correlation between the brain gene expression profiles of severe COVID-19 patients showing increased expression of innate immune response genes and genes implicated in AD pathogenesis. The use of a mouse-adapted strain of SARS-CoV-2 (MA10) in an aged mouse model shows evidence of viral neurotropism, prolonged viral infection, increased expression of tau aggregator FKBP51, interferon-inducible gene Ifi204, and complement genes C4 and C5AR1. Brain histopathology shows AD signatures including increased tau-phosphorylation, tau-oligomerization, and α-synuclein expression in aged MA10 infected mice. The results of gene expression profiling of SARS-CoV-2-infected and AD brains and studies in the MA10 aged mouse model taken together, for the first time provide evidence that SARS-CoV-2 infection alters expression of genes in the brain associated with the development of AD thus indicating that common molecular markers in SARS-CoV-2 infection and AD can be useful for developing novel therapies targeting AD and related neurologic disorders.

In an embodiment, a method of treating a neurodegenerative disease in a patient previously infected with a respiratory virus is presented comprising: obtaining or having obtained a sample from the patient; obtaining or having obtained an expression level of at least one gene or one gene product in the sample wherein the at least one gene or the at least one gene product is selected from the group consisting of CCL20, CTCFL, CXCL8, EGFR, GFAP, IFI-16, IL-17, IL-18, IL-6R, KLF4, LGALS3, TAC1, CAV1, FKBP5, HSP90, HSPA8, IFITM3, C3/4, C5AR1, CR1, CALB1, CAMKK2, BDNF, CCK/BR, PLAT, MAPT, APP, STAT3, Calbindin, NLRP3, and combinations thereof; comparing the expression level of the at least one gene or gene expression product to a predetermined control expression level; and administering a therapeutic agent capable of modulating expression of the at least one gene or the at least one gene product if the expression level of the at least one gene or the at least one gene product is increased as compared to the control level.

The respiratory virus may be severe acute respiratory syndrome coronavirus 2 (SARS CoV-2). The patient may have been diagnosed with long covid previously.

The at least one gene or at least one gene product may be selected from the group consisting of FKBP5, IFITM3, IFI16, CR1, C5AR1, and NLRP3. At least one of FKBP51, NLRP3, or IFI16 may be upregulated as compared to a control. The therapeutic agent may be an RNA interference agent, such as an shRNA, siRNA or anti-sense oligonucleotide, targeting the at least one gene or gene expression product selected from FKBP51, NLRP3 or IFI16. The RNA interference agent may be encapsulated within or complexed to an outer surface of a nanoparticle. In some embodiments, the nanoparticle is a dendrimer nanoformulation with the RNA interference agent complexed to the outer surface of the dendrimer nanoformulation. The therapeutic agent may be administered intranasally.

The neurodegenerative disease may be selected from the group consisting of Alzheimer's disease (AD), Parkinson's disease (PD), corticobasal degeneration (CBD), progressive supranuclear palsy (PSP), sporadic frontotemporal dementia (FTD), frontotemporal lobar degeneration (FTLD), Lytico-bodig disease (Guam Parkinson-dementia complex), HIV-associated Dementia, multiple sclerosis, amyotrophic lateral sclerosis (ALS), dementias, encephalopathy, and Pick's Disease. In some embodiments, the neurodegenerative disease is Alzheimer's disease. The patient may be an aged patient.

In an embodiment, a method of preventing a neurodegenerative disease in a patient previously infected with a respiratory virus is presented comprising: obtaining or having obtained a sample from the patient; obtaining or having obtained an expression level of at least one gene or one gene product in the sample wherein the at least one gene or the at least one gene product is selected from the group consisting of CCL20, CTCFL, CXCL8, EGFR, GFAP, IFI-16, IL-17, IL-18, IL-6R, KLF4, LGALS3, TAC1, CAV1, FKBP5, HSP90, HSPA8, IFITM3, C3/4, C5AR1, CR1, CALB1, CAMKK2, BDNF, CCK/BR, PLAT, MAPT, APP, STAT3, Calbindin, NLRP3, and combinations thereof; comparing the expression level of the at least one gene or gene expression product to a predetermined control expression level; and administering a therapeutic agent capable of modulating expression of the at least one gene or the at least one gene product if the expression level of the at least one gene or the at least one gene product is increased as compared to the control level.

The respiratory virus may be severe acute respiratory syndrome coronavirus 2 (SARS CoV-2). The patient may have been diagnosed with long covid previously.

The at least one gene or at least one gene product may be selected from the group consisting of FKBP5, IFITM3, IFI16, CR1, C5AR1, and NLRP3. At least one of FKBP51, NLRP3, or IFI16 may be upregulated as compared to a control. The therapeutic agent may be an RNA interference agent, such as an shRNA, siRNA or anti-sense oligonucleotide, targeting the at least one gene or gene expression product selected from FKBP51, NLRP3 or IFI16. The RNA interference agent may be encapsulated within or complexed to an outer surface of a nanoparticle. In some embodiments, the nanoparticle is a dendrimer nanoformulation with the RNA interference agent complexed to the outer surface of the dendrimer nanoformulation. The therapeutic agent may be administered intranasally.

The neurodegenerative disease may be selected from the group consisting of Alzheimer's disease (AD), Parkinson's disease (PD), corticobasal degeneration (CBD), progressive supranuclear palsy (PSP), sporadic frontotemporal dementia (FTD), frontotemporal lobar degeneration (FTLD), Lytico-bodig disease (Guam Parkinson-dementia complex), HIV-associated Dementia, multiple sclerosis, amyotrophic lateral sclerosis (ALS), dementias, encephalopathy, and Pick's Disease. In some embodiments, the neurodegenerative disease is Alzheimer's disease or dementia. The patient may be an aged patient.

In a further embodiment, a method of reducing tauopathy in a patient in need thereof is presented comprising: obtaining or having obtained a sample from the patient; obtaining or having obtained an expression level of at least one gene or one gene product in the sample wherein the at least one gene or the at least one gene product is selected from the group consisting of CCL20, CTCFL, CXCL8, EGFR, GFAP, IFI-16, IL-17, IL-18, IL-6R, KLF4, LGALS3, TAC1, CAV1, FKBP5, HSP90, HSPA8, IFITM3, C3/4, C5AR1, CR1, CALB1, CAMKK2, BDNF, CCK/BR, PLAT, MAPT, APP, STAT3, Calbindin, NLRP3, and combinations thereof; comparing the expression level of the at least one gene or gene expression product to a predetermined control expression level; and administering a therapeutic agent capable of modulating expression of the at least one gene or the at least one gene product if the expression level of the at least one gene or the at least one gene product is increased as compared to the control level wherein modulation of the at least one gene or at least one gene product reduces the tauopathy.

The respiratory virus may be severe acute respiratory syndrome coronavirus 2 (SARS CoV-2). The patient may have been diagnosed with long covid previously.

The at least one gene or at least one gene product may be selected from the group consisting of FKBP5, IFITM3, IFI16, CR1, C5AR1, and NLRP3. At least one of FKBP51, NLRP3, or IFI16 may be upregulated as compared to a control. The therapeutic agent may be an RNA interference agent, such as an shRNA, siRNA or anti-sense oligonucleotide, targeting the at least one gene or gene expression product selected from FKBP51, NLRP3 or IFI16. The RNA interference agent may be encapsulated within or complexed to an outer surface of a nanoparticle. In some embodiments, the nanoparticle is a dendrimer nanoformulation with the RNA interference agent complexed to the outer surface of the dendrimer nanoformulation. The therapeutic agent may be administered intranasally.

The neurodegenerative disease may be selected from the group consisting of Alzheimer's disease (AD), Parkinson's disease (PD), corticobasal degeneration (CBD), progressive supranuclear palsy (PSP), sporadic frontotemporal dementia (FTD), frontotemporal lobar degeneration (FTLD), Lytico-bodig disease (Guam Parkinson-dementia complex), HIV-associated Dementia, multiple sclerosis, amyotrophic lateral sclerosis (ALS), dementias, encephalopathy, and Pick's Disease. In some embodiments, the neurodegenerative disease is Alzheimer's disease. The patient may be an aged patient.

### BRIEF DESCRIPTION OF THE DRAWINGS

For a fuller understanding of the invention, reference should be made to the following detailed description, taken in connection with the accompanying drawings, in which:

FIG. 1 is a table depicting ADRD risk/pathology genes.

FIG. 2A-C are a series of images depicting transcriptomic changes observed in the frontal cortex of severe COVID and AD brains (A) Mean diff plot showing significantly upregulated (red) and downregulated (green) genes in AD brains versus control brains. (B) MA plot showing significantly upregulated (red) and downregulated (green) genes in COVID-19 versus control brains. (C) Differentially expressed genes in each dataset sorted into Gene Ontology (GO) pathways (Panther-db). Pathways containing at least 10 differentially expressed genes from each dataset are shown. A complete gene list is shown in FIGS. 3A-B.

FIG. 3A is a table listing differentially expressed genes in Covid-19.

FIG. 3B is a table listing differentially expressed genes in AD.

FIG. 4A-B are a series of images depicting dimensionality reduction and clustering of individual patient samples. (A) Uniform Manifold Approximation and Projection (UMAP) clustering of individual AD vs control sample. (B) Principle component (PC) clustering of individual COVID-19 vs control samples.

FIG. 5 is a table depicting the fold change and p value of ADRD pathology genes in COVID-19 brains.

FIG. 6 are a series of graphs depicting immune cell types present in COVID and control brains as estimated from sequencing data with CIBERSORT.

FIG. 7 is a table depicting hub genes identified in protein interaction network of COVID brain samples using STRING database and CytoHubba.

FIG. 8 is an image depicting severe COVID alters expression of key Alzheimer's risk genes in the human brain Network of AD risk genes depicting experimentally observed interactions (IPA database) and fold change of gene expression observed in COVID frontal cortex samples versus control, GEO: GSE188847 (green/red).

FIG. 9A-C are a series of images depicting infection with SARS-CoV-2 MA10 virus induces neuroinflammation in aged mice (A) Representative images of plaque assays performed on homogenized brain tissue collected from MA10-infected 3-, 6-, and 20-month-old mice. (B) Histogram depicting quantification of viral plaque assay to determine the concentration of infectious particles present in brain tissue (PFU/mL). (C) Histogram depicting mRNA fold change of CoV-2 nucleoprotein (N) expression in brains collected from MA10-infected mice compared with mock.

FIG. 9D is a series of histograms depicting mRNA fold change of proinflammatory genes (I16, Tnf-α, and Ccl20), and inflammasome genes (Nlrp3, Ifi204, Ilb), in infected brain. n=3; data expressed as mean±SEM; * compared with respective mock; #18 DPI compared with 4 DPI of the same brain region; *, #p<0.05 by ANOVA with Holm-Sidak test.

FIG. 10 is a series of images depicting detection of negative strand CoV2 viral RNA in brain sections using FISH probes. Representative confocal images showing the co-localization (yellow, marked by arrows) of negative strand CoV2 RNA (RFP labeled) with NeuN (Neuron)-(GFP) in brain sections of 17 months old mice, 14 dpi. Scale bar 20 μm, magnified 10 μm.

FIG. 11 is a series of images depicting SARS-CoV-2 infection induces Casp1(p20) expression in Aged Mice. Immunostaining and Histogram showing Caspase1 expression in infected brain-olfactory bulb, cortex, hippocampus, n=3/group, data expressed as mean±SEM,* Compared to respective mock, *p<0.05, **p<0.005.

FIG. 12A is an image depicting infection with SARS-CoV-2 MA10 virus induces ADRD risk genes and complement activation in aged mice. (A) Histograms depicting mRNA fold change of ADRD risk genes (Fkbp5, Ifitm3), and complement activation genes (Cr1, C5ar1), in brains collected fromMA10-infected mice.

FIG. 12B-C are a series of images depicting infection with SARS-CoV-2 MA10 virus induces ADRD risk genes and complement activation in aged mice. (B) Bright-field images and respective quantification showing FKBP5 expression in the olfactory bulb, cortex, and hippocampus of MA10-infected 20-month-old mice brains at 18 DPI. (C) Ingenuity Pathway Analysis (IPA) network showing genes that are differentially expressed in cortex of infected aged mice (20 months old) 18 DPI as assayed by nCounter neuroinflammatory and glial profiling panel. n=3; data expressed as mean±SEM; * compared with respective mock; #compared with respective 4 DPI; *p<0.05 by ANOVA with Holm-Sidak test.

FIG. 13 is an image depicting the network of AD risk genes depicting experimentally observed gene interactions (IPA) in brain cortex of infected aged (20 mo-old) mice. Fold changes of gene expression at 18 days post infection compared to mock were examined by nCounter neuroinflammatory and glial panels or by qPCR. and data were compiled into a single network. The proinflammatory genes (I16, Tnfa, and Ccl20), and inflammasome genes (Nlrp3, Ifi204, IlB) were assayed by qPCR and the remaining genes were assayed by nCounter (n=3).

FIG. 14A is a series of images depicting SARS-CoV-2 MA10 infection induces p-tau expression in olfactory bulb, cortex, and hippocampus of 6- and 20-month-old mice. (A) Bright-field images showing immunoperoxidase staining of p-tau (pT231) in olfactory bulb (top panel), cortex (middle panel), and hippocampus (bottom panel) of 3-, 6-, and 20-month-old mice at 18 DPI. Histogram showing ImgaeJ quantification of p-tau expressing cells.

FIG. 14B is a series of images depicting SARS-CoV-2 MA10 infection induces p-tau expression in olfactory bulb, cortex, and hippocampus of 6- and 20-month-old mice. (B) Bright-field images showing immunoperoxidase staining of tau (T22), oligomeric expression in olfactory bulb (top panel), cortex (middle panel), and hippocampus (bottom panel) of 3-, 6- and 20-month old mice, at 18 DPI. Histogram showing ImgaeJ quantification of tau (T22)-expressing cells.

FIG. 14C is a series of images depicting SARS-CoV-2 MA10 infection induces p-tau expression in olfactory bulb, cortex, and hippocampus of 6- and 20-month-old mice. (C) Co-localization of CD31-p-tau expression in olfactory bulb, cortex, and hippocampus. CD31: red, DAPI: blue, and p-tau: green. Arrows indicate areas of p-tau/CD31 co-localization. n=3; data expressed as mean±SEM; * compared with respective mock; #compared between 6- and 20-month-old infected mice, *, #p<0.05.

FIG. 15A-B are a series of images depicting CD31 and VWF staining of MA10 infected mouse brains: C57Bl/6 3, 6 and 20 month old mice were infected with SARS CoV2 MA10 virus-100k PFU. (A) Representative images showing the expression of CD31 (marker for endothelial cells) and VWF (von Willebrand factor marker for vascular damage) in olfactory bulb. Upper panel: co localization of CD31 (RFP) and VWF (GFP) depicting vascular damage shown by arrows, lower panel: CD31 (RFP) and VWF (GFP). (B) Histogram representing the quantification of VWF+ vascular coverage (% VWF expression with respect to CD31 expression). n=3, Data expressed as mean±SEM, * Compared to control ##, *p<0.05, **p<0.005.

FIG. 16A-B are a series of images depicting SARS-CoV-2 MA 10 infection induces gliosis in olfactory bulb, cortex and hippocampus of 6 and 20 Months aged mice. (A) Immunostaining showing GFAP/DAPI staining in infected brain-olfactory bulb (upper panel), cortex (middle panel) and hippocampus (lower panel) at 18 DPI, (B) histogram representing quantification of GFAP immunoreactivity (Intden/unit area), n=3/group, data expressed as mean±SEM,* Compared to respective mock, #Compared to infection *, #p<0.05, **, ##p<0.005.

FIG. 17A-B are a series of images depicting SARS-CoV-2 MA 10 infection induces gliosis in olfactory bulb, cortex and hippocampus of 6 and 20 Months aged mice. (A) Immunostaining showing IBA1 (red)/DAPI staining in infected brain-olfactory bulb (upper panel), cortex (middle panel) and hippocampus (lower panel), at 18 DPI, (B) histogram representing quantification of IBA1+ cells, n=3/group, data expressed as mean±SEM,* Compared to respective #mock, #Compared to infection *, p<0.05, **, ##p<0.005, ***.

FIG. 18A-B are a series of images depicting SARS-CoV-2 infection induces demyelination in Aged Mice. 3, 6 and 20 months aged C57Bl/6 mice were infected with SARS CoV2 MA10 virus-100k PFU. The infected mice were sacrificed 18 days post infection. (A) Immunostaining showing MBP staining in infected brain-Striatum, (B) quantification of immunostaining (MBP intensity), n=3/group, data expressed as mean±SEM, * Compared to respective mock, ## Compared to infection *, p<0.05, **, ##p<0.005.

FIG. 19 is a series of images depicting SARS-CoV-2 infection induces α-synuclein expression in Aged Mice. 3, 6 and 20 month old C57Bl/6 mice were infected with SARS CoV2 MA10 virus, Immunostaining showing αSyn staining in infected brain-olfactory bulb, cortex and hippocampus, quantification of immunostaining at 18 DPI, n=3/group, data expressed # as mean±SEM,* Compared to respective mock, #Compared to infection *, p<0.05.

FIG. 20A-B are a series of images depicting Infection with MA10 virus increases IFI204 and FKBP51 expression and circulating t-Tau in aged mice. Brightfield images of immunoperoxidase staining and respective quantification in the OB, cortex and hippocampus of MA10 infected brains of 6-month-old (6 M) and 20-month-old (20 M) C57BL/6NJ mice at 18 dpi shown. (A) Histogram showing NearCYTE quantification of IFI204 expression (% Area) and (B) ImageJ quantification (Intden/unit area) of FKBP51 expression. N=3; Data expressed as mean±SEM, Student t-test. *p<0.05.

FIG. 21A-E are a series of images depicting infection with MA10 virus induces p-Tau expression and circulating t-Tau in 6- and 20-month-old mice. Brightfield images of immunoperoxidase staining and respective quantification in the OB, cortex and hippocampus of MA10 infected brains of 3-month (3M), 6-month-old (6 M) and 20-month-old (20 M) C57BL/6NJ mice at 18 dpi shown. (A-B) Histogram showing ImageJ quantification of p-Tau expressing cells, and (C-D) ImageJ quantification of Tau (T22)-expressing cells. N=3; Data expressed as mean±SEM, Student t-test. *p<0.05. (E) Circulating t-Tau in serum of 3-, 6-, and 20-month-old mice at 18 dpi measured by ELISA using a highly sensitive mouse Tau ELISA Kit (ABclonal). M: Mock; Inf: Infected; n=3; data expressed as mean±SEM, Student t-test. *p<0.05.

FIG. 22A-C are a series of images depicting CoV2-MA10 infection increases expression of t-Tau, pTau and oligomeric Tau in adult PS19 mice. Brightfield images of immunoperoxidase staining and respective quantification in the OB, frontal cortex and hippocampus of PS19 infected brains at 18 dpi shown. (A) t-Tau, (B) pTau 231 and (C) (T22) Tau. n=Mock (3), Infected (4). Histogram showing NearCYTE quantification of t-Tau, p-tau and T22. Data expressed as mean±SEM, Student t-test. *p<0.05

FIG. 23A-E are a series of images depicting CoV2-MA10 infection increases neuroinflammation and FKBP51 in adult PS19 mice. Brightfield images of immunoperoxidase staining, fluorescent images and respective quantification in the OB, frontal cortex and hippocampus of PS19 infected brains at 18 dpi shown. (A) CoV2-N protein, (B) IFI204, (C) GFAP, (D) IBA1, (E) FKBP51 Image panels show representative images (20×) of OB, prefrontal cortex and hippocampus of mock and infected mice. n=Mock (3), Infected (4). Histogram showing NearCYTE quantification of CoV2-N, IFI204, FKBP51 and ImageJ quantification of GFAP and IBA1. Data expressed as mean±SEM, Student t-test. *p<0.05

FIG. 24A-C is a series of images depicting DPX-pshFkbp5 decreases FKBP51, pTau and oligomeric Tau in adult MA10-infected mice. Brightfield images of immunoperoxidase staining and respective quantification in the OB, cortex and hippocampus of MA10 infected brains at 18 dpi shown. A) FKBP51, B) phospho (pT231) and C) oligomeric (T22) tau. Image panels show presentative images (20×) of OB, prefrontal cortex and hippocampus of infected and treated mice. n=Mock (5), Control (4), shScr (4), shFkbp5 (5). Histogram showing NearCYTE quantification of FKBP51, p-tau and T22. Data expressed as mean±SEM, Student t-test. *, #p<0.05

FIG. 25A-B are a series of images depicting DPX-pshFkbp5 decreases GFAP and IBA1 expression in adult MA10-infected mice. Immunofluorescent images of and respective quantification in the OB, cortex and hippocampus of MA10 infected brains at 18 dpi shown. A) GFAP, and B) IBA1. Image panels show representative images (20×) of OB, prefrontal cortex and hippocampus of infected and treated mice. n=Mock (5), Control (4), shScr (4), shFkbp5 (5). Histogram showing ImageJ quantification of GFAP and IBA1. Data expressed as mean±SEM, Student t-test. ***p<0.05.

FIG. 26 is an image depicting treatment of silfi204 or siNlrp3 significantly reduced FKBP51 expression in CoV2-MA10 infected HT22 cells. Representative Western blot of CoV2-MA10 infected HT22 cells treated as indicated. Densitometry analysis for FKBP51 normalized by Actin from two biological replicates. **p=0.01, ***p=0.001, by one-way ANOVA.

### DETAILED DESCRIPTION OF THE PREFERRED EMBODIMENT

In the following detailed description of the preferred embodiments, reference is made to the accompanying drawings, which form a part hereof, and within which are shown by way of illustration specific embodiments by which the invention may be practiced. It is to be understood that other embodiments may be utilized, and structural changes may be made without departing from the scope of the invention.

### Definitions

Unless otherwise defined, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are described herein. All publications mentioned herein are incorporated herein by reference in their entirety to disclose and describe the methods and/or materials in connection with which the publications are cited. It is understood that the present disclosure supercedes any disclosure of an incorporated publication to the extent there is a contradiction.

As used herein, the singular forms “a,” “an” and “the” include plural referents unless the context clearly dictates otherwise. For example, “a nanoparticle” includes “nanoparticles” or “plurality of nanoparticles”.

As used in this specification and the appended claims, the term “or” is generally employed in its sense including “and/or” unless the context clearly dictates otherwise.

All numerical designations, such as pH, temperature, time, concentration, and molecular weight, including ranges, are approximations which are varied up or down by increments of 1.0, 0.1, 0.01 or 0.001 as appropriate. It is to be understood, even if it is not always explicitly stated that all numerical designations are preceded by the term “about”. It is also to be understood, even if it is not always explicitly stated, that the reagents described herein are merely exemplary and that equivalents of such are known in the art and can be substituted for the reagents explicitly stated herein.

Concentrations, amounts, solubilities, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include the individual values and sub-ranges within the indicated range. Thus, included in this numerical range are individual values such as 2, 3, and 4 and sub-ranges such as from 1-3, from 2-4 and from 3-5, etc. This same principle applies to ranges reciting only one numerical value. Furthermore, such an interpretation should apply regardless of the range or the characteristics being described.

As used herein, the term “comprising” is intended to mean that the products, compositions, and methods include the referenced components or steps, but not excluding others. “Consisting essentially of” when used to define products, compositions, and methods, shall mean excluding other components or steps of any essential significance. Thus, a composition consisting essentially of the recited components would not exclude trace contaminants and pharmaceutically acceptable carriers. “Consisting of” shall mean excluding more than trace elements of other components or steps.

As used herein, “about” means approximately or nearly and in the context of a numerical value or range set forth means±15% of the numerical.

As used herein “patient” is used to describe a mammal, preferably a human, to whom treatment is administered, including prophylactic treatment with the compositions of the present invention. Non-limiting examples of mammals include humans, rodents, aquatic mammals, domestic animals such as dogs and cats, farm animals such as sheep, pigs, cows and horses. “Patient” and “subject” are used interchangeably herein. In some embodiments, the patient is an aged patient. “Aged” as used herein refers to a mature adult mammal. For example, mice that are at least 6 months old are considered aged while humans above about age 40 may be considered aged.

The genes of the present invention may serve as biomarkers for: (1) the diagnosis of or susceptibility to disease; (2) the prognosis of diseases (e.g. monitoring disease progression or regression from one biological state to another); or (3) the evaluation of the efficacy to a treatment for disease. For the diagnosis of or susceptibility to disease, the level of the specific gene in the subject can be compared to a baseline or control level in which if the level is above the control level, a certain disease is implicated. The prognosis of disease can be assessed by comparing the level of the specific gene biomarker at a first timepoint to the level of the biomarker at a second timepoint which occurs at a given interval after the first timepoint. The evaluation of the efficacy of the treatment for a disease can be assessed by comparing the level of the specific gene biomarker at a first timepoint before administration of the treatment to the level of the biomarker at a second timepoint which occurs at a specified interval after the administration of the treatment.

The term “biomarker” is used herein to refer to a molecule whose level of nucleic acid or protein product has a quantitatively differential concentration or level with respect to an aspect of a biological state of a subject. “Biomarker” is used interchangeably with “marker” herein. The level of the biomarker can be measured at both the nucleic acid level as well as the polypeptide level. At the nucleic acid level, a nucleic acid gene or a transcript which is transcribed from any part of the subject's chromosomal and extrachromosomal genome, including for example the mitochondrial genome, may be measured. Preferably an RNA transcript, more preferably an RNA transcript includes a primary transcript, a spliced transcript, an alternatively spliced transcript, or an mRNA of the biomarker is measured. At the polypeptide level, a pre-propeptide, a propeptide, a mature peptide or a secreted peptide of the biomarker may be measured. A biomarker can be used either solely or in conjunction with one or more other identified biomarkers so as to allow correlation to the biological state of interest as defined herein. Specific examples of biomarkers covered by the present invention include genes involved in inflammation, protein folding/trafficking, complement activation, calcium homeostasis, and amyloid/tau processing. More specifically, biomarkers covered by the present invention includes genes CCL20, CTCFL, CXCL8, EGFR, GFAP, IFI-16 (mouse homolog IFI204), IL-17, IL-18, IL-6R, KLF4, LGALS3, TAC1, CAV1, FKBP5, HSP90, HSPA8, IFITM3, C3/4, C5AR1, CR1, CALB1, CAMKK2, BDNF, CCK/BR, PLAT, MAPT, APP, STAT3, Calbindin, and NLRP3.

The term “expression level” as used herein refers to detecting the amount or level of expression of a biomarker of the present invention. The act of actually detecting the expression level of a biomarker refers to the act of actively determining whether a biomarker is expressed in a sample or not. This act can include determining whether the biomarker expression is upregulated, downregulated or substantially unchanged as compared to a control level expressed in a sample. The expression level in some cases may refer to detecting transcription of the gene encoding a biomarker protein and/or to detecting translation of the biomarker protein.

Expression of genes/transcripts and/or polypeptides encoded by the genes represented by the biomarkers of the present invention can be measured by any of a variety of methods known in the art. In general, expression of a nucleic acid molecule (e.g. RNA or DNA) can be detected by any suitable method or technique of measuring or detecting gene or polynucleotide sequence or expression. Such methods include, but are not limited to, polymerase chain reaction (PCR), reverse transcriptase PCR (RT-PCR), in situ PCR, quantitative PCR (q-PCR), in situ hybridization, Southern blot, Northern blot, sequence analysis, microarray analysis, detection of a reporter gene, or any other DNA/RNA hybridization platforms.

The term “quantifying” or “quantitating” when used in the context of quantifying transcription levels of a gene can refer to absolute or relative quantification. Absolute quantification can be achieved by including known concentration(s) of one or more target nucleic acids and referencing the hybridization intensity of unknowns with the known target nucleic acids (e.g. through the generation of a standard curve). Alternatively, relative quantification can be achieved by comparison of hybridization signals between two or more genes, or between two or more treatments to quantify the changes in hybridization intensity and, by implication transcription level.

The term “differential expression” as used herein refers to qualitative or quantitative differences in the temporal and/or spatial gene expression patterns within and among cells and tissues. A differentially expressed gene may qualitatively have its expression altered, including an activation or inactivation, such as in normal versus diseased tissue. Genes may be turned off or on in a given state relative to another state thus allowing comparison of two or more states. A qualitatively regulated gene may exhibit an expression pattern within a state or cell type that can be detectable by standard techniques. Alternatively, the difference in expression may be quantitative such that expression of the gene is modulated, up-regulated (resulting in an increased amount of transcript), or down-regulated (resulting in a decreased amount of transcript). The degree to which expression varies needs to be large enough to quantify via standard characterization techniques such as expression arrays, quantitative reverse transcriptase PCR, Northern blot analysis, real-time PCR, in situ hybridization, and RNase protection.

The term “expression profile” as used herein refers to a genomic expression profile, for example an expression profile of microRNAs or proteins. The profiles may be generated by any means for determining a level of a nucleic acid sequence, e.g. quantitative hybridization of microRNA, labeled microRNA, amplified microRNA, cDNA, quantitative PCR, ELISA for quantitation, etc. For proteins, the profiles may be generated by any means for determining a level of a protein, e.g. Western blot, immunoblot, enzyme-linked immunosorbent assay (ELISA), radioimmunoassay (RIA), immunoprecipitation, surface plasmon resonance, chemiluminescence, fluorescent polarization, phosphorescence, immunohistochemical analysis, liquid chromatography mass spectrometry (LC-MS), matrix-assisted laser desorption/ionization time-of-flight (MALDI-TOF), mass spectrometry, microcytometry, microarray, microscopy, fluorescence activated cell sorting (FACS), flow cytometry, and assays based on a property of the protein including but not limited to DNA binding, ligand binding, or interaction with other protein partners. The profile must allow for the analysis of differential gene expression between two samples.

The terms “overexpression” and “underexpression” as used herein refers to the expression of a gene of a patient at a greater or lesser level, respectively, than the normal or control expression of the gene, as measured by gene expression product expression such as mRNA or protein expression, in a sample that is greater than the standard of error of the assay used to assess the expression. A “significant” expression level may be a level which either meets or is above or below a predetermined score for a gene.

“Sample,” as used herein, refers to a composition that is obtained or derived from a subject and/or individual of interest that contains a cellular and/or other molecular entity that is to be characterized and/or identified, for example, based on physical, biochemical, chemical, and/or physiological characteristics. For example, the phrase “disease sample” and variations thereof refers to any sample obtained from a subject of interest that would be expected or is known to contain the cellular and/or molecular entity that is to be characterized. Samples include, but are not limited to, tissue samples, primary or cultured cells or cell lines, cell supernatants, cell lysates, platelets, serum, plasma, vitreous fluid, lymph fluid, synovial fluid, follicular fluid, seminal fluid, amniotic fluid, milk, whole blood, blood-derived cells, urine, cerebro-spinal fluid, saliva, sputum, tears, perspiration, mucus, tumor lysates, and tissue culture medium, tissue extracts such as homogenized tissue, tumor tissue, cellular extracts, and combinations thereof.

“Tissue sample” or “cell sample” as used herein refers to a collection of similar cells obtained from a tissue of a subject or individual. The source of the tissue or cell sample may be solid tissue as from a fresh, frozen and/or preserved organ, tissue sample, biopsy, and/or aspirate; blood or any blood constituents such as plasma; bodily fluids such as cerebral spinal fluid, amniotic fluid, peritoneal fluid, or interstitial fluid; cells from any time in gestation or development of the subject. The tissue sample may also be primary or cultured cells or cell lines. Optionally, the tissue or cell sample is obtained from a disease tissue/organ. For instance, a “tumor sample” is a tissue sample obtained from a tumor or other cancerous tissue. The tissue sample may contain a mixed population of cell types (e.g., tumor cells and non-tumor cells, cancerous cells and non-cancerous cells). The tissue sample may contain compounds which are not naturally intermixed with the tissue in nature such as preservatives, anticoagulants, buffers, fixatives, nutrients, antibiotics, or the like.

A “reference sample,” “reference cell,” “reference tissue,” “control sample,” “control cell,” or “control tissue,” as used herein, refers to a sample, cell, tissue, standard, or level that is used for comparison purposes. In one embodiment, a reference sample, reference cell, reference tissue, control sample, control cell, or control tissue is obtained from a healthy and/or non-diseased part of the body (e.g., tissue or cells) of the same subject or individual. For example, the reference sample, reference cell, reference tissue, control sample, control cell, or control tissue may be healthy and/or non-diseased cells or tissue adjacent to the diseased cells or tissue (e.g., cells or tissue adjacent to a tumor). In another embodiment, a reference sample is obtained from an untreated tissue and/or cell of the body of the same subject or individual. In yet another embodiment, a reference sample, reference cell, reference tissue, control sample, control cell, or control tissue is obtained from a healthy and/or non-diseased part of the body (e.g., tissues or cells) of an individual who is not the subject or individual. In even another embodiment, a reference sample, reference cell, reference tissue, control sample, control cell, or control tissue is obtained from an untreated tissue and/or cell of the body of an individual who is not the subject or individual.

The term “gene expression product” or “expression product” as used herein refers to an RNA transcribed from a gene (either pre- or post-processing) or an amino acid (e.g. a polypeptide, protein, or peptide regardless of any secondary modifications, such as glycosylation, lipidation or phosphorylation) encoded by the gene and generated by the gene when the gene is transcribed (either pre- or post-modification) and translated. An agent is said to increase gene expression if the application of a therapeutically effective amount of the agent to a cell or subject results in an increase in either an RNA or polypeptide expression product or both. An agent is said to decrease gene expression if the application of a therapeutically effective amount of the agent to a cell or subject results in a decrease in either an RNA or polypeptide expression product or both.

The term “nucleic acid” as used herein may be double-stranded, single-stranded, or contain portions of both double and single stranded sequence. If the nucleic acid is single-stranded, the sequence of the other strand is also identifiable and thus the definition includes the complement of the sequence disclosed. Nucleic acids include one or more types of: polydeoxyribonucleotides (containing 2-deoxy-D-ribose), polyribonucleotides (containing D-ribose), and any other type of polynucleotide that is an N-glycoside of a purine or pyrimidine base, or modified purine or pyrimidine bases (including abasic sites). The term “nucleic acid,” as used herein, also includes polymers of ribonucleosides or deoxyribonucleosides that are covalently bonded, typically by phosphodiester linkages between subunits, but in some cases by phosphorothioates, methylphosphonates, and the like. “Nucleic acids” include single- and double-stranded DNA, as well as single- and double-stranded RNA. Exemplary nucleic acids include, without limitation, gDNA; hnRNA; mRNA; rRNA, tRNA, micro RNA (miRNA), small interfering RNA (siRNA), small nucleolar RNA (snORNA), small nuclear RNA (snRNA), short hairpin (sh) RNA, and small temporal RNA (stRNA), and the like, and any combination thereof.

“Administering” or “administration” as used herein refers to the process by which the compositions of the present invention are delivered to the patient. The compositions may be administered in various ways, including but not limited to, orally, nasally, and parenterally. “Parenteral administration” as used herein refers to modes of administration other than enteral and topical administration, usually by injection, and includes, but is not limited to, intravenous, intramuscular, intraarterial, intrathecal, intracapsular, intraorbital, intracardiac, intradermal, intraperitoneal, transtracheal, subcutaneous, subcuticular, intra-articular, subcapsular, intrathecal, intraventricular, intracisternal, intranigral, subarachnoid, intraspinal, and intrasternal injection and infusion. Dosing can be by any suitable route, e.g., by injections, such as intravenous or subcutaneous injections, depending in part on whether the administration is brief or chronic. Various dosing schedules including but not limited to single or multiple administrations over various time-points, bolus administration, and pulse infusion are contemplated herein.

A “therapeutic agent” as used herein refers to a substance, composition, compound, chemical, component or extract that has measurable specified or selective physiological activity when administered to an individual in a therapeutically effective amount. In some embodiments, the therapeutic agent may be a nucleic acid. In some embodiments, the nucleic acid is an RNA interference agent that inhibits expression of the selected gene(s). Such RNA interference agents include, but are not limited to, small interfering RNA (siRNA), short hairpin (sh) RNA, and anti-sense oligonucleotide for the gene(s). At least one therapeutic agent is used in the compositions of the present invention, however in some embodiments, multiple therapeutic agents are used. In some embodiments, one or more therapeutic agents may be encapsulated within a nanoparticle or complexed to an outer surface of a nanoparticle.

A “therapeutically effective amount” as used herein is defined as concentrations or amounts of components which are sufficient to effect beneficial or desired clinical results, including, but not limited to, any one or more of treating symptoms of neurologic diseases, particularly neurodegenerative diseases, more particularly Alzheimer's disease and related dementias (ADRD). Compositions of the present invention can be used to effect a favorable change in the condition whether that change is an improvement, such as stopping, reversing, or reducing symptoms of neurologic diseases, or a complete elimination of symptoms due to neurologic diseases. In accordance with the present invention, a suitable single dose size is a dose that is capable of preventing or alleviating (reducing or eliminating) a symptom in a patient when administered one or more times over a suitable time period. One of skill in the art can readily determine appropriate single dose sizes for systemic administration based on the size of the animal and the route of administration. The dose may be adjusted according to response.

The dosing of compounds and compositions to obtain a therapeutic or prophylactic effect is determined by the circumstances of the patient, as known in the art. The dosing of a patient herein may be accomplished through individual or unit doses of the compounds or compositions herein or by a combined or prepackaged or pre-formulated dose of a compounds or compositions. An average 20-30 g mouse has a brain weighing about 0.4-0.5 g. An average 400 g rat has a brain weighing 2 g. An average human brain weighs 1508 g, which can be used to direct the amount of therapeutic needed or useful to accomplish the treatment described herein.

The amount of the compound in the drug composition will depend on absorption, distribution, metabolism, and excretion rates of the drug as well as other factors known to those of skill in the art. Dosage values may also vary with the severity of the condition to be alleviated. The compounds may be administered once, or may be divided and administered over intervals of time. It is to be understood that administration may be adjusted according to individual need and professional judgment of a person administrating or supervising the administration of the compounds used in the present invention.

The dose of the compounds administered to a subject may vary with the particular composition, the method of administration, and the particular disorder being treated. The dose should be sufficient to affect a desirable response, such as a therapeutic or prophylactic response against a particular disorder or condition. It is contemplated that one of ordinary skill in the art can determine and administer the appropriate dosage of compounds disclosed in the current invention according to the foregoing considerations.

Dosing frequency for the composition includes, but is not limited to, at least about once every three weeks, once every two weeks, once a week, twice a week, three times a week, four times a week, five times a week, six times a week, or daily. In some embodiments, the interval between each administration is less than about a week, such as less than about any of 6, 5, 4, 3, 2, or 1 day. In some embodiments, the interval between each administration is constant. For example, the administration can be carried out daily, every two days, every three days, every four days, every five days, or weekly. In some embodiments, the administration can be carried out twice daily, three times daily, or more frequently. Administration can also be continuous and adjusted to maintaining a level of the compound within any desired and specified range.

The administration of the composition can be extended over an extended period of time, such as from about a month or shorter up to about three years or longer. For example, the dosing regimen can be extended over a period of any of about 2, 3, 4, 5, 6, 7, 8, 9, 10, 11, 12, 18, 24, 30, and 36 months. In some embodiments, there is no break in the dosing schedule. In some embodiments, the interval between each administration is no more than about a week.

The compounds used in the present invention may be administered individually, or in combination with or concurrently with one or more other compounds used against neurodegenerative diseases. Additionally, compounds used in the present invention may be administered in combination with or concurrently with other therapeutics for neurodegenerative diseases.

“Prevention” or “preventing” or “prophylactic treatment” as used herein refers to any of: halting the effects of neurodegenerative disease, reducing the effects of neurodegenerative disease, reducing the incidence of neurodegenerative disease, reducing the development of neurodegenerative disease, delaying the onset of symptoms of neurodegenerative disease, increasing the time to onset of symptoms of neurodegenerative disease, and reducing the risk of development of neurodegenerative disease. In some embodiments, the neurodegenerative disease is Alzheimer's disease or related dementias.

“Treatment” or “treating” as used herein refers to any of the alleviation, amelioration, elimination and/or stabilization of a symptom, as well as delay in progression of a symptom of a particular disorder. For example, “treatment” of neurodegenerative disease may include any one or more of the following: amelioration and/or elimination of one or more symptoms associated with neurodegenerative disease, reduction of one or more symptoms of neurodegenerative disease, stabilization of symptoms of neurodegenerative disease, and delay in progression of one or more symptoms of neurodegenerative disease. Treatment may include reduction of tau accumulation. In some embodiments, the neurodegenerative disease is Alzheimer's disease or related dementias.

“Infection” as used herein refers to the invasion of one or more microorganisms such as bacteria, viruses, fungi, yeast, or parasites in the body of a patient in which they are not normally present. In certain embodiments, the infection is from a respiratory virus such as a respiratory syncytial virus, Influenza virus, or coronavirus. In some embodiments, the coronavirus is severe acute respiratory syndrome coronavirus 2 (SARS-CoV-2). Other coronaviruses contemplated herein include, but are not limited to, severe acute respiratory syndrome coronavirus 1 (SARS-CoV-1), Middle East respiratory syndrome coronavirus (MERS-CoV), human coronavirus OC43 (HcoV-OC43), human coronavirus 229E (HcoV-229E), porcine deltacoronavirus (PDCoV) (porcine), infectious bronchitis virus (IBV, avian), and other coronaviruses of pandemic potential including Delta coronavirus, duvinacovirus, Embecovirus, Gammacoronavirus, Merbecovirus, Nobecovirus and Sarbecovirus.

The pharmaceutical compositions of the subject invention can be formulated according to known methods for preparing pharmaceutically useful compositions. Furthermore, as used herein, the phrase “pharmaceutically acceptable carrier” means any of the standard pharmaceutically acceptable carriers. The pharmaceutically acceptable carrier can include diluents, adjuvants, and vehicles, as well as implant carriers, and inert, non-toxic solid or liquid fillers, diluents, or encapsulating material that does not react with the active ingredients of the invention. Examples include, but are not limited to, phosphate buffered saline, physiological saline, water, and emulsions, such as oil/water emulsions. The carrier can be a solvent or dispersing medium containing, for example, ethanol, polyol (for example, glycerol, propylene glycol, liquid polyethylene glycol, and the like), suitable mixtures thereof, and vegetable oils. Formulations are described in a number of sources that are well known and readily available to those skilled in the art. For example, Remington's Pharmaceutical Sciences (Martin E W Easton Pennsylvania, Mack Publishing Company, 19^{th} ed.) describes formulations which can be used in connection with the subject invention.

For ease of administration, the subject compounds may be formulated into various pharmaceutical forms. As appropriate compositions there may be cited all compositions usually employed for systemically or topically administering drugs. To prepare the pharmaceutical compositions of this invention, atranorin or other polyphenolic lichen acid isolate, as the active ingredient is combined in intimate admixture with a pharmaceutically acceptable carrier, which may take a wide variety of forms depending on the form of preparation desired for administration. These pharmaceutical compositions are desirably in unitary dosage form suitable, preferably, for administration nasally, orally, rectally, percutaneously, or by parenteral injection. For example, in preparing the compositions in oral dosage form, any of the usual pharmaceutical media may be employed, such as, for example, water, glycols, oils, alcohols and the like in the case of oral liquid preparations such as suspensions, syrups, elixirs and solutions; or solid carriers such as starches, sugars, kaolin, lubricants, binders, disintegrating agents and the like in the case of powders, pills, capsules and tablets. Because of their ease in administration, tablets and capsules often represent the most advantageous oral dosage unit form, in which case solid pharmaceutical carriers are obviously employed. For parenteral compositions, the carrier will usually comprise sterile water, at least in large part, though other ingredients, for example, to aid solubility, may be included. Injectable solutions, for example, may be prepared in which the carrier comprises saline solution, glucose solution or a mixture of saline and glucose solution.

“Nanoparticle” as used herein refers to a particle or structure which is biocompatible with and sufficiently resistant to chemical and/or physical destruction by the environment of such use so that a sufficient number of the nanoparticles remain substantially intact after delivery to the site of application or treatment and whose size is in the nanometer range. For the purposes of the present invention, a nanoparticle typically ranges between about 1 nm to about 1000 nm, preferably between about 50 nm and about 500 nm, more preferably between about 50 nm and about 350 nm, more preferably between about 100 nm and about 250 nm. As used herein, the term “nanoparticle” includes, but is not limited to, dendrimers, micelles, polymeric nanoparticles, liposomes, niosomes, transferosome, liponanoparticle, lipid nanoparticles, nanostructured lipid nanocarriers (NLC), solid lipid nanoparticles (SLN), hybrid lipid-polymer nanoparticles, bicelle, polymerosomes, lamellar structures, and lipid vesicles, among other delivery systems that can be used suitably to deliver an therapeutic agent.

“Dendrimer” as used herein refers to a highly branched synthetic polymer macromolecules capable of being used in delivery of therapeutic agents, such as drugs, in a patient. Dendrimers as used herein refer to nanoparticle dendrimers having a size less than 250 nm and preferably having a size of 100 nm or less. Dendrimers are constructed by the successive addition of branching group layers with each branching group layer being a new generation. Dendrimers capable of being used herein include, but are not limited to, Poly(propyleneimine) dendrimers (PPI), Poly(amidoamine) dendrimers (PAMAM), Poly 2,2-bis(methylol)propionic acid (PBisMPA), Poly(benzyl ether) dendrimers (PBzE), poly(lysine) dendrimers (PLL), and polymelamine (triazine) dendrimers. If PAMAM dendrimers are used, any of generations 0-11 (G0-G11) may be used. In some embodiments, PAMAM G4 is used. PAMAM dendrimers generally comprise an ethylenediamine core, a repetitive branching amidoamine internal structure and a primary amine terminal surface.

“Dendriplex” as used herein refers to dendrimers that have been functionalized by the complexation (conjugation) of at least one therapeutic agents and/or genes to deliver genes/gene products and/or therapeutic agents to the brain. In some embodiments, the dendrimers have an shRNA encoding DNA plasmid complexed to the outer surface of the dendrimer.

“Short hairpin RNA” or “shRNA” as used herein refers to single stranded RNA molecules that are constructed by connecting sense and antisense strands of an siRNA duplex with a loop sequence thus allowing a single transcript to fold back on a duplex structure upon being transcribed. After transcription, the shRNA molecules are processed into siRNAs by the Dicer enzyme and are capable of suppressing a gene.

“Neurodegenerative disease” as used herein refers to a disease which is caused by damage to the central nervous system and can be identified by neuronal death. Further, the term “neurodegenerative disease” as used herein describes neurodegenerative diseases which are associated with tauopathies. Exemplary neurodegenerative diseases include Alzheimer's disease (AD), Parkinson's disease (PD), corticobasal degeneration (CBD), progressive supranuclear palsy (PSP), sporadic frontotemporal dementia (FTD), frontotemporal lobar degeneration (FTLD), Lytico-bodig disease (Guam Parkinson-dementia complex), HIV-associated Dementia, multiple sclerosis, amyotrophic lateral sclerosis (ALS), other dementias including those related to Alzheimer's disease, encephalopathy, and Pick's Disease.

“Tauopathies” as used herein refers to a class of neurodegenerative diseases caused by misfolding of the tau protein which results in the deposition of abnormal tau (tubulin associated unit) in the brain. These aggregates form neurofibrillary tangles that can lead to neuronal toxicity and degeneration. Tauopathies can include both movement and cognitive/behavioral disorders or non-specific amnesic symptoms in advanced age.

“Long Covid” as used herein refers to patients exhibiting COVID-19 symptoms for weeks or months despite testing negative for the virus. These long-haul symptoms can include neurological symptoms, such as brain fog, anosmia, ageusia, insomnia, and headaches; debilitating fatigue, body aches, and joint pain; respiratory symptoms such as coughing and shortness of breath; and/or cardiovascular symptoms such as persistent pain or pressure in the chest, tachycardia, dizziness, and heart palpitations.

A comparison of gene expression changes in brains of human patients with AD or COVID-19 versus their respective controls along with brain pathological studies in the CoV-2 MA10 aged mouse model has led to identification of several common hub genes and pathways that are similar in pathogenesis of AD in mice and humans. This analysis is the first of its kind wherein transcript expression in the brain (cortex) has been compared between elderly patients with severe COVID-19 and patients with AD. Previously, a solely bioinformatic study compared the AD brain transcript profile (GEO: GSE147507; n=97) with COVID lung gene expression (GEO: GSE132903; n=2).⁷²

The mouse studies using CoV-2-MA10 virus revealed that CoV-2 neurotropism was dependent upon the age of the mice. Thus, replication of MA10 virus was evident in the brains of aged (6- and 20-month-old) mice but not in young 3-month-old mice. Notably, despite evidence of neurotropism for SARS-CoV and Middle East respiratory syndrome (MERS)-CoV and affirmative CoV-2 neurotropism studies in vitro in human cells,⁷³ in vivo neurotropism of CoV-2 has remained unclear due to the lack of virus detection in many post-mortem patient brain and CNS samples. The establishment of CoV-2-MA10 infection in an aged mouse model has provided the first in vivo evidence for CoV-2 neurotropism in wild-type mice. The MA10 aged mouse model has provided a unique opportunity to investigate the connection between viral infection and ADRDs including the mechanistic underpinnings of this relationship. With millions of patients infected by CoV-2 having mild, moderate, and severe infections and 15%-30% of those also suffering from long COVID, potential increased risk of ADRDs would have significant clinical impact.¹⁵

The major evidence for SARS-CoV-2 neurotropism (N-gene transcript expression and viral plaque assay) definitively demonstrates viral replication in the brain tissue, though the cellular localization of the virus within the mouse brain (endothelial, microglia, neuron, etc.) and the extent of viral replication within these cells remain unknown. The findings are consistent with the previous epidemiological and laboratory reports of severe viral infections leading to a preponderance of CNS diseases including AD, Parkinson's disease, and encephalopathy.⁶⁻⁸ Notably, the inventors observed increased vWF expression in aged, infected brains as evidence of vascular damage along with increased p-tau in regions adjacent to the endothelial cell marker CD31, suggesting there may be a vascular etiology to this pathology. This is consistent with primate data that suggested that CoV-2 replication is mainly within vascular endothelial cells in the brain.⁷⁴ Further, the inventors found that changes in expression of genes in COVID-19 brains included the Wnt signaling pathway. This pathway is dysregulated in AD, with beta catenin (CTNNB1) aggregation linked to proteosomal dysfunction and tau phosphorylation by GSK3b,⁷⁵ thus contributing to AD risk.

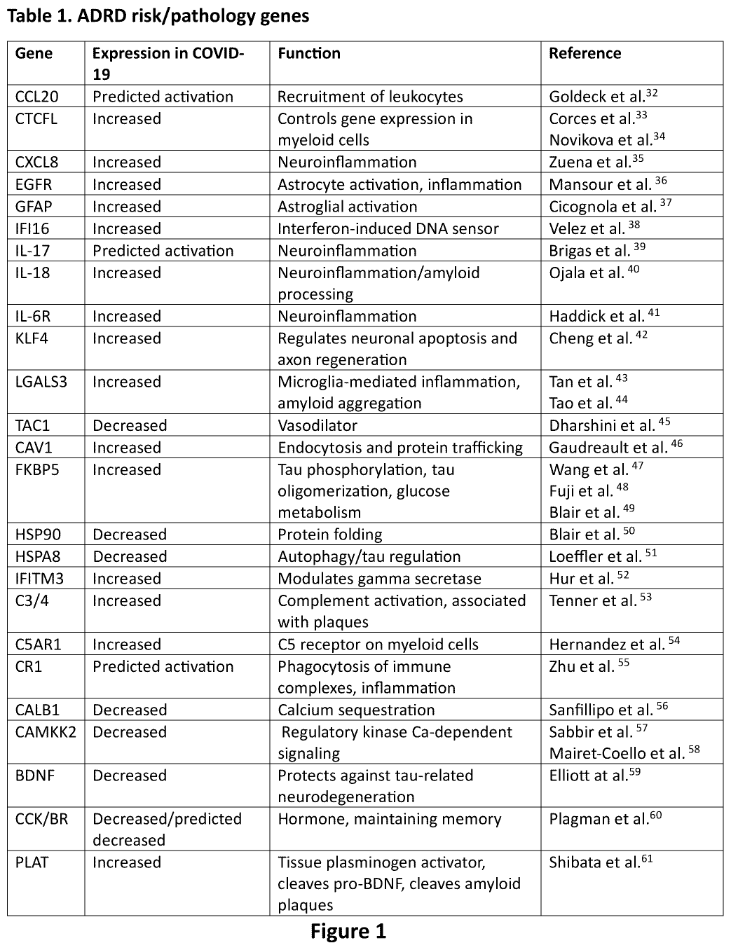

In a broad sense, the CoV-2 neuropathology contains many elements overlapping with AD. Neuroinflammation is a major driver of both diseases; however, additional pleiotropic signaling pathways are also implicated, including EGFR, Wnt, and immune cell activation. The inventors have observed gene expression changes within these pathways in both diseases, albeit with only moderate overlap between individual genes. Increased oxidative stress and dysregulated ribosomal function are known features of both diseases as well. Through pathway analysis of known AD-gene relationships in the IPA and STRING databases and literature review, the inventors have grouped ADRD risk genes into 5 categories: inflammation, protein folding/trafficking, complement activation, calcium homeostasis, and amyloid/tau processing (FIG. 1). Many changes in expression of these risk genes and specific gene mutations have been associated with late-stage AD through sequencing of brain tissue from post-mortem patients. However, due to the complexity of the disease and the inability to collect longitudinal samples, direct causes of AD remain poorly defined. Recently, the once-controversial “infectious hypothesis” of AD has been gaining more attention.^{76,77} Although it seems unlikely that an infectious agent such as a prion or virus will emerge as the direct cause of AD, it is possible that the molecular signaling changes brought on by inflammation due to aging and followed by infection (“double-hit model”) could serve as a trigger for disease progression in individuals who are already genetically predisposed. The data presented herein support this hypothesis.

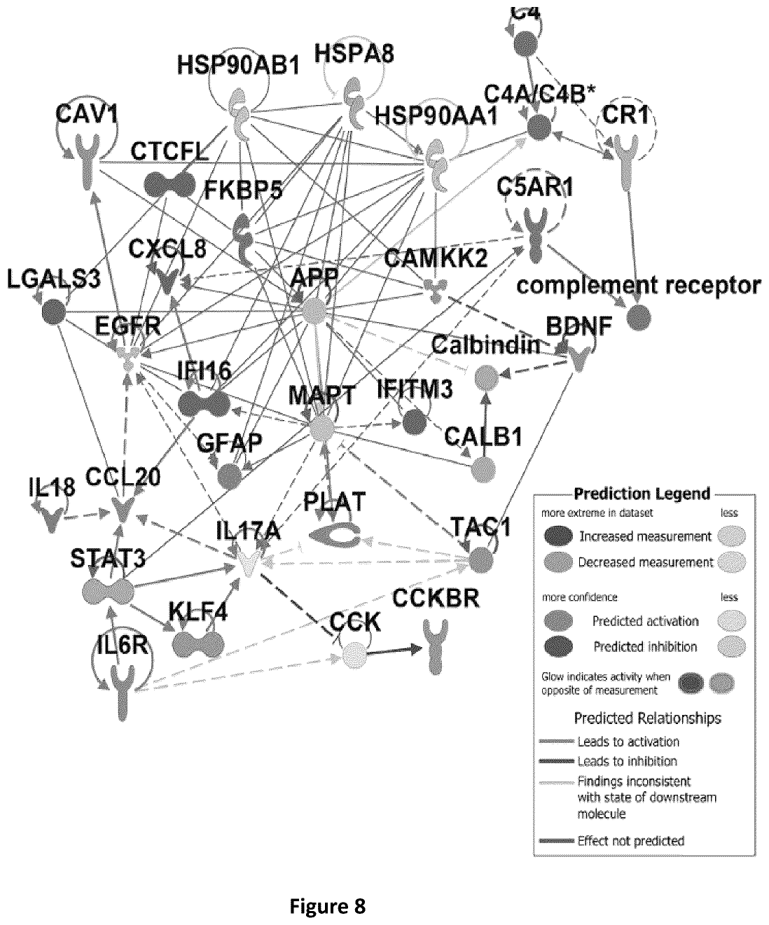

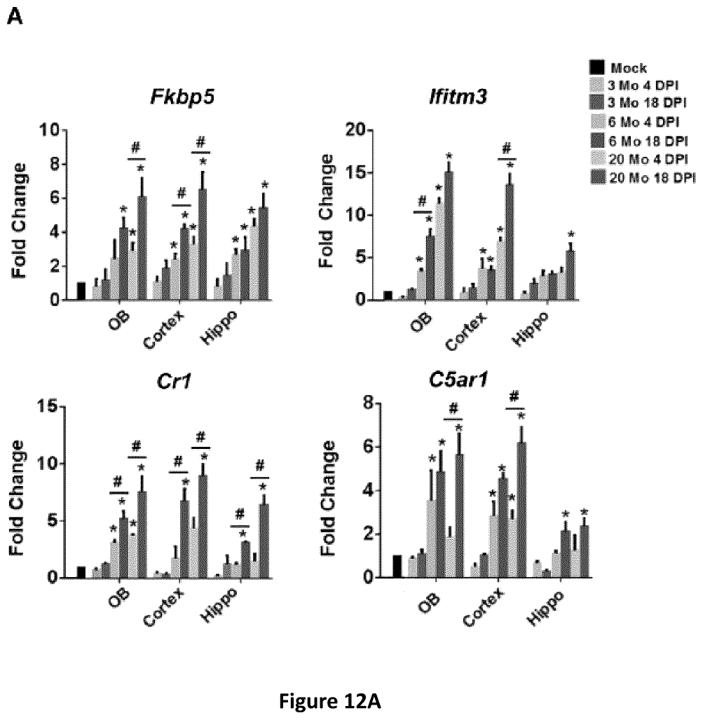

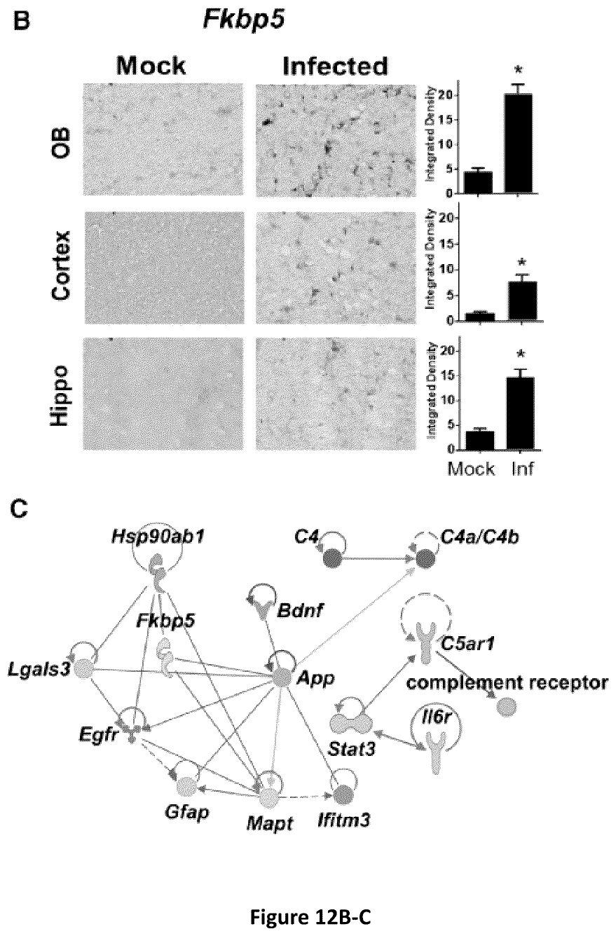

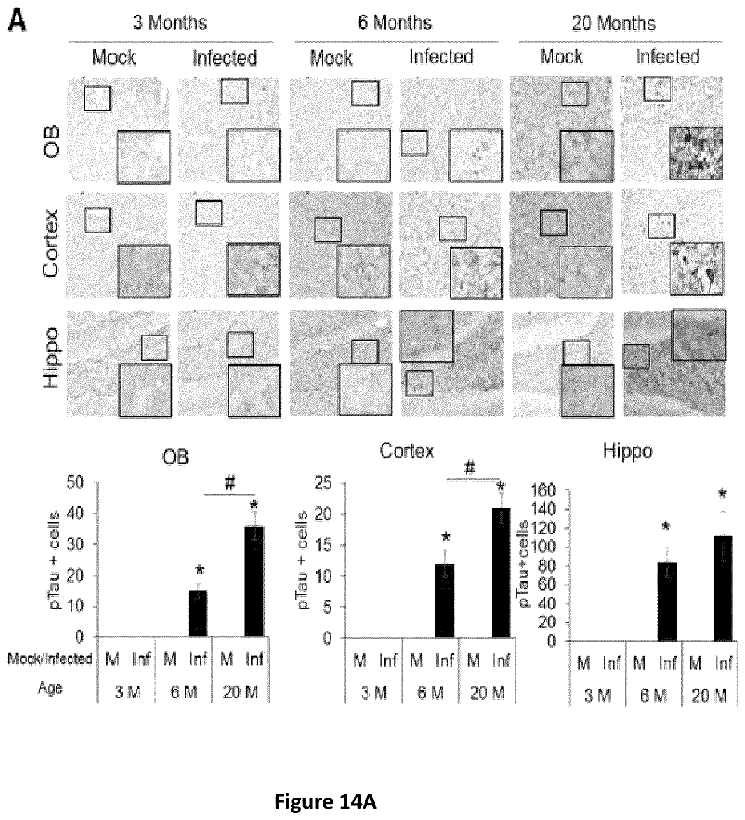

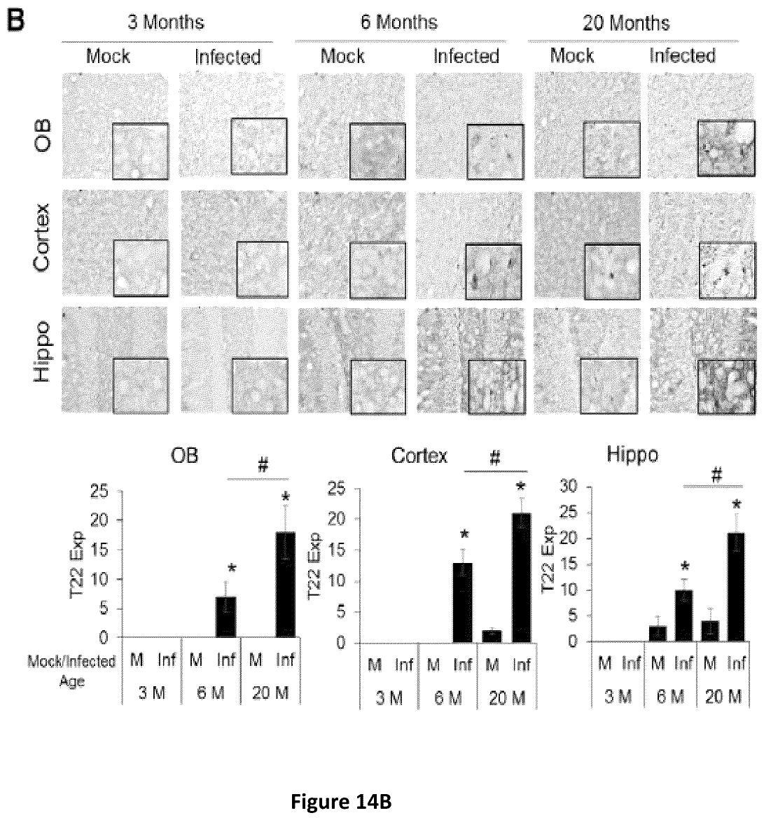

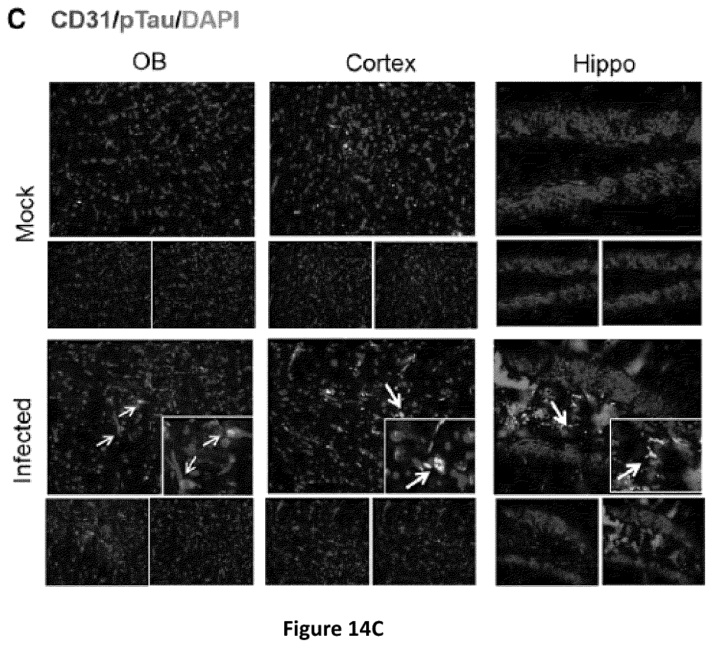

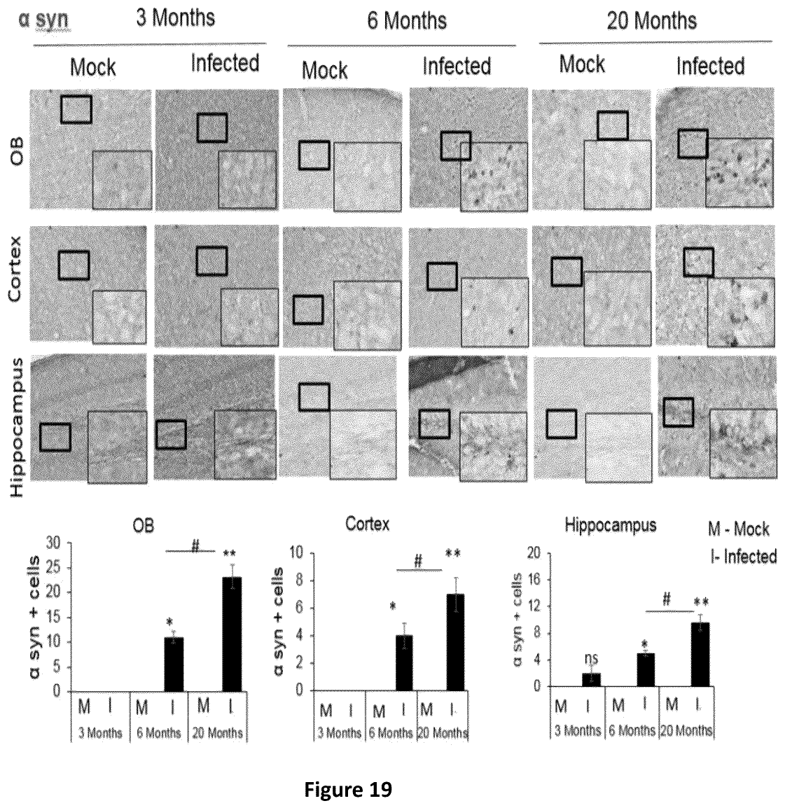

Consistent with the inventors previous report,²⁹ the results of the gene expression profiles in CoV-2-infected and AD brains showed similarly increased activation of complement/coagulation signaling and the inflammasome, which are known hallmarks of moderate and severe COVID-19. The inventors have presented evidence of this same activation in both human and mouse brains (IL-18, CCL20, NLRP3, C4a, and C5AR1) (FIGS. 8A and 12A-C). Furthermore, the inventors have shown how increased expression of these genes may activate downstream AD risk genes (IFI16, IFITM3, FKBP5, GFAP) ultimately leading to tau and α-synuclein pathology as observed in the mouse model (FIGS. 14 and 19). Indeed, some of these genes play dual roles in innate immunity and AD. IFITM3 is an interferon-stimulated gene that prevents viral entry into cells by disrupting cholesterol synthesis and shuttling viral particles to lysosomes. However, it is also known to have secondary activities including activation of PI3K signaling and activation of γ-secretase to increase Aβ production. α synuclein-GSK3β activity could exacerbate this AD pathology by promoting a feedback loop of tau phosphorylation, and aggregation of both tau and Aβ, with a simultaneous increase in Aβ production.⁷⁸ Presenilin-1 (PSEN1) plays a role in proteolytic cleavage of APP to Aβ, which interacts with α-synuclein, forming complexes as witnessed in patients with AD with PSEN1 mutations. Altogether, there are a number of in vitro and in vivo studies supporting the leading role that α-synuclein plays in several mechanisms and processes linked to AD.⁷⁹⁻⁸¹

Perhaps the most significant connection between CoV-2 infection and AD observed in both human tissue and the mouse model is the potential for CoV-2 infection to promote tau phosphorylation and oligomerization. This phenomenon has been observed at the protein level in the brain tissue of both elderly dementia patients and patients who have died from COVID-19.66 In this case, a leaky calcium channel driven by virus-induced inflammasome activation and oxidative stress is hypothesized to promote tau pathology through a reduction in calbindin. The inventors observed a similar decrease in calbindin expression in COVID-19 brains along with a decrease in CAMKK2 and evidence of inflammasome activation (increased IL-18, IFI16, and STAT3). In the mouse model, the inventors observed increased Nlrp3, Tnf-α, and active cleaved caspase-1 (p20), indicating inflammasome activation, but the inventors were also able to observe the tau pathology directly, including significant increases in tau phosphorylation and oligomerization. Studies are ongoing to determine the timeline of inflammasome induction in the CoV-2-infected brain and whether this induction is dependent on recognition of viral RNA or host DNA damage.

Overall, the inventors have shown that CoV-2 infection induces gene expression changes in multiple pathways linked to AD susceptibility including inflammation, protein chaperones, amyloid processing, ubiquitin mediated protein degradation, FKBPs in tau processing, endoplasmic reticulum (ER) protein transport, and calcium homeostasis. The inventors observed the significant overlap in the gene expression profiles of the two diseases across multiple functional pathways in combination with the ADRD pathology observed in the brains of aged, infected mice which can help determine targets against CoV2-induced tauopathies.

### Results

Gene Expression Changes in Brains of Patients with COVID-19 and AD

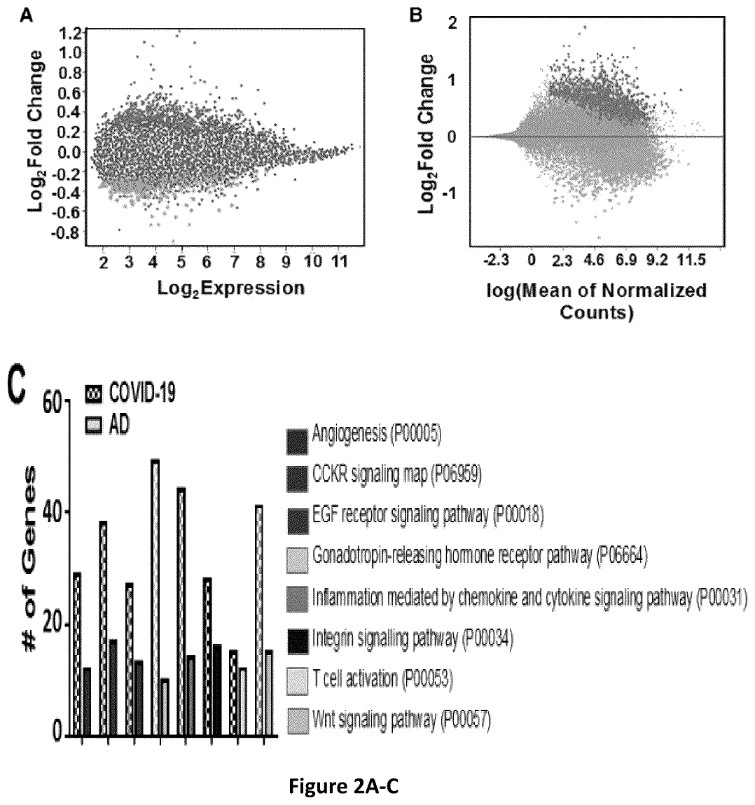

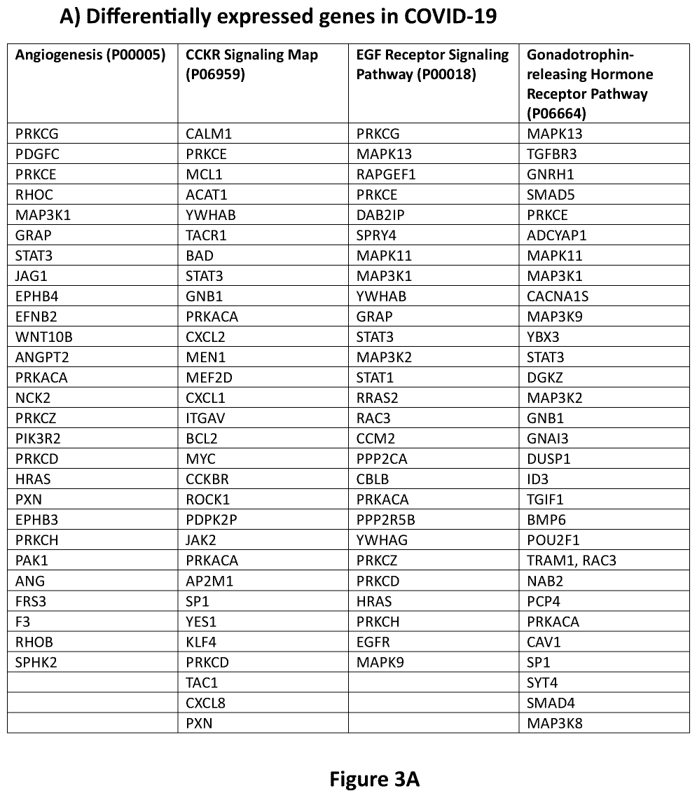

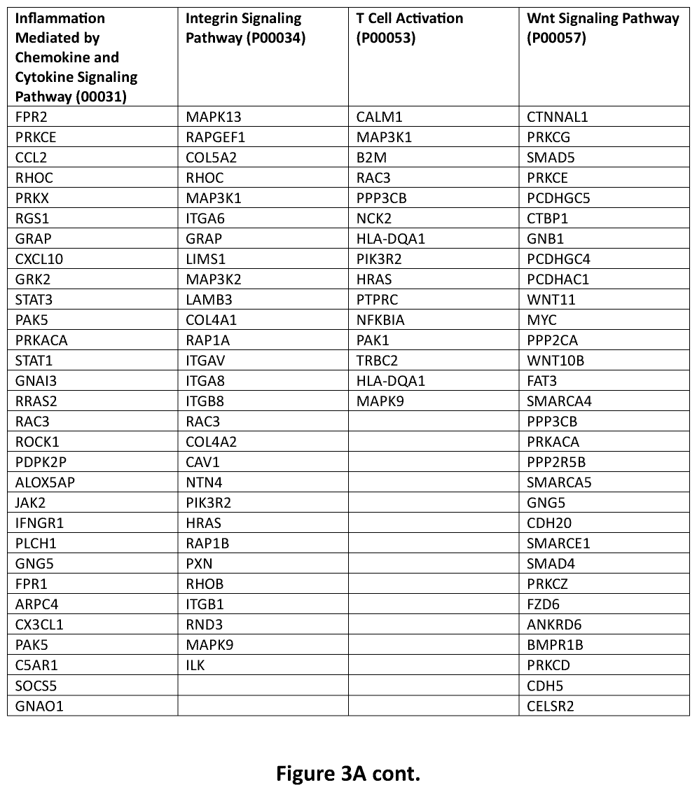

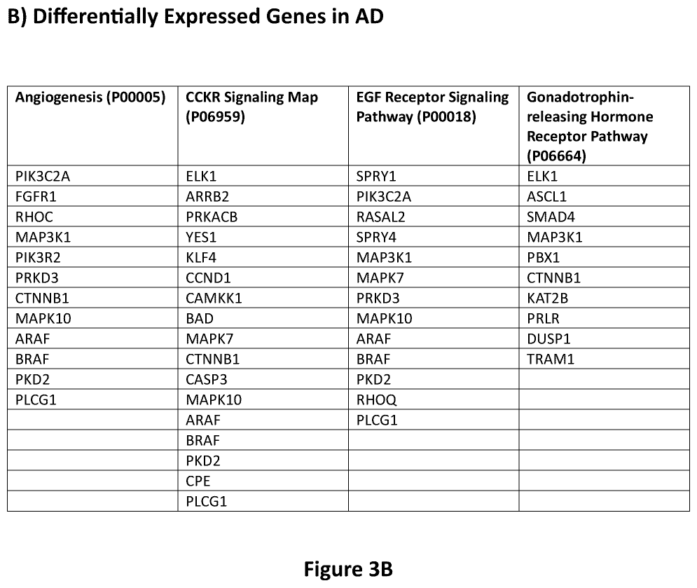

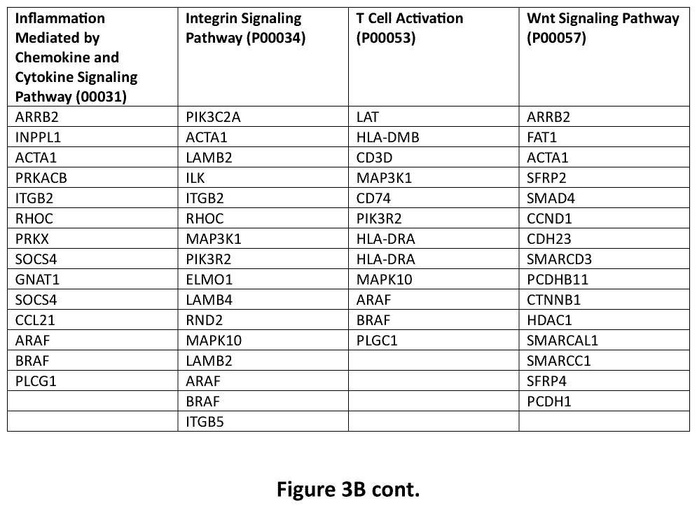

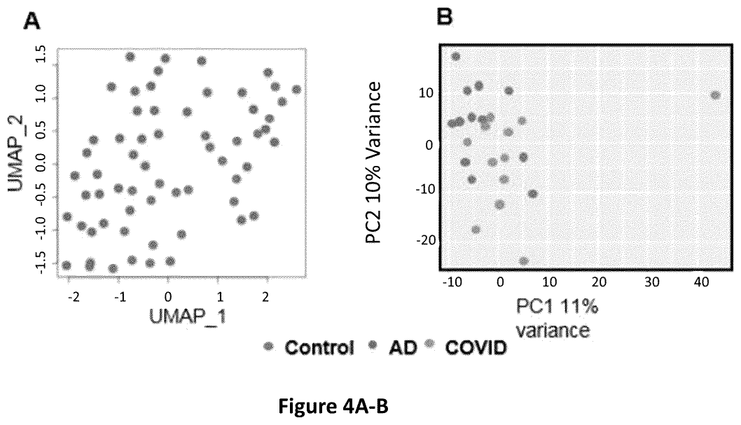

The inventors obtained whole-transcriptome expression data from COVID-19 versus control and AD versus control frontal cortex patient samples from the NCBI GEO database. Differential gene analysis generated 2,446 genes significantly up- or downregulated in the brains of COVID-19 patients and 856 genes significantly up- or downregulated in the brains of patients with AD (FIGS. 2A and B). Dimensionality reduction and clustering yielded only moderate separation between disease and control groups, highlighting the impact of patient-to-patient variation in gene expression (FIGS. 4A and B). Sorting the differentially expressed genes into GO pathways revealed several common pathways affected by both diseases, although the absolute gene counts were skewed by the difference in size of each dataset (FIG. 2C). A full listing of the differentially expressed genes is shown in FIGS. 3A-B.

### COVID-19 Brains Exhibit an AD Risk Gene Expression Signature

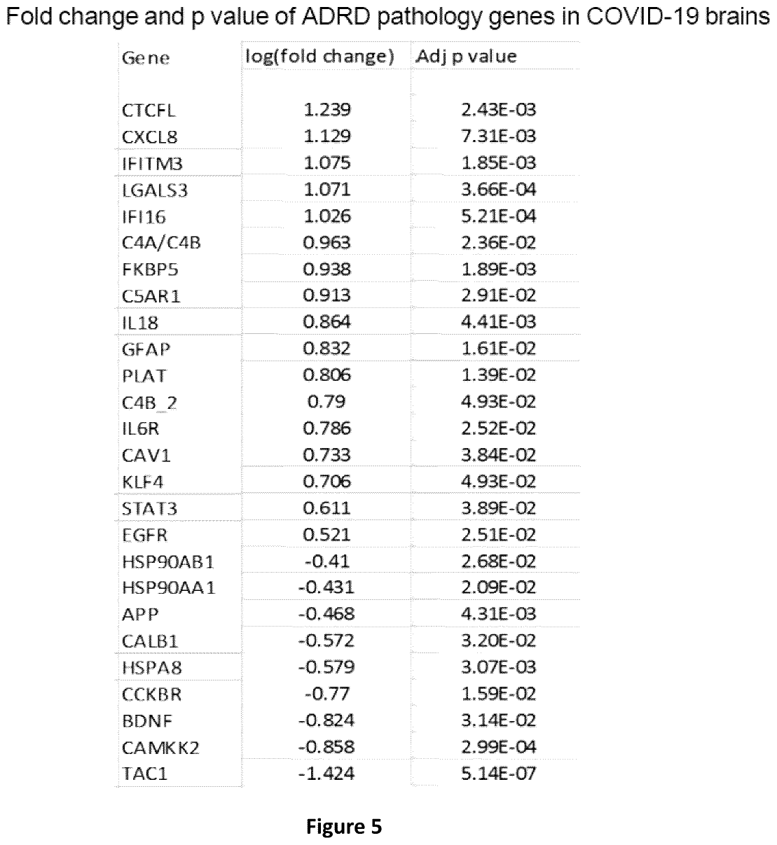

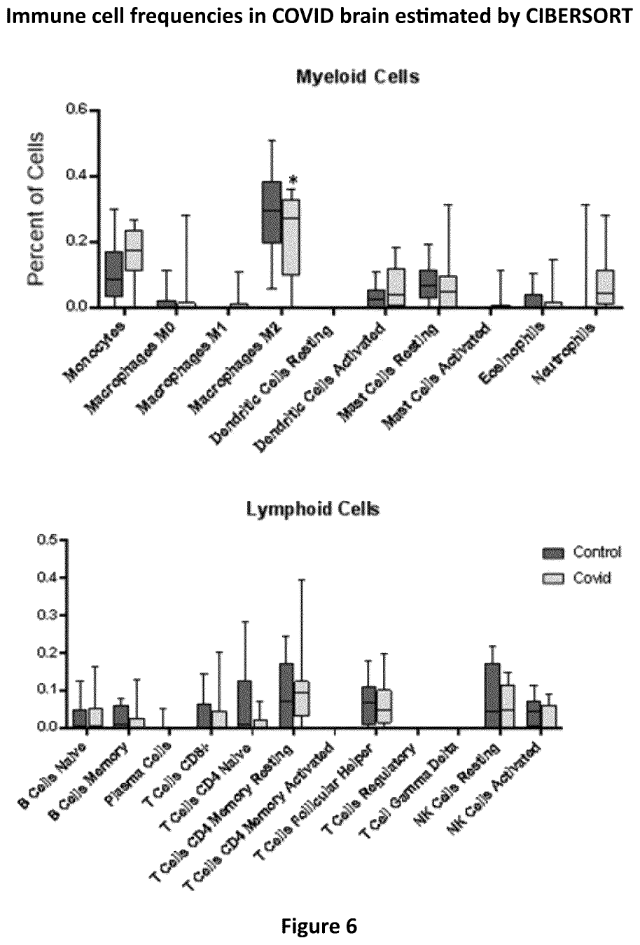

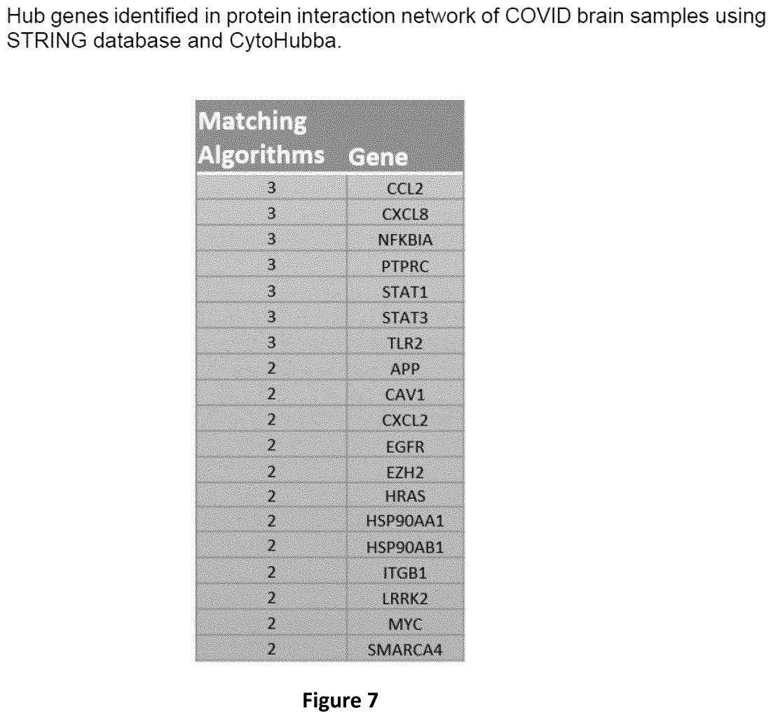

In order to compile a set of AD risk genes, the inventors searched relevant literature for studies describing the molecular etiology of AD and providing evidence for the specific function of individual genes/proteins in AD onset or progression. The inventors compiled the findings into five functional categories: inflammation, protein folding/trafficking, complement activation, calcium homeostasis, and amyloid/tau processing (FIG. 1). This gene list was imported into Ingenuity Pathway Analysis (IPA) and plotted experimentally observed interactions as a network. Fold change values (FIG. 5) from the COVID-19 brain dataset were overlaid onto the network, revealing increased inflammatory cytokines IL-18, CXCL8, and IL-6 receptor along with the transcription factors STAT3 and KLF4 and the marker of neuroinflammation GFAP. Although specific cell types responsible for promoting inflammation in these patients is not known, the inventors did observe an increase in gene signatures associated with monocytes, neutrophils, and activated dendritic cells in the COVID-19 brains; however, these changes were not statistically significant (FIG. 6). Hub gene analysis using the Search Tool for the Retrieval of Interacting Genes/Proteins (STRING) database of protein interactions reinforced the evidence of neuroinflammation yielding canonical mediators and regulators of inflammation (CCL2, CXCL8, NF-kB, and STAT3) as some of the differentially expressed genes with the most interactors in the dataset (FIG. 7). Complement activation was also evident from increased C4a and C5AR1. Decreased expression of HSP chaperones HSP90AB1, HSP90AA1, and HSPA8 was observed, while FKBP5 was increased. Calcium signaling components (CAMKK2 and calbindin) and the receptor for cholecystokinin, CCKBR, were all decreased in expression (FIG. 8).

### SARS-CoV-2 MA10 Neurotropism is Increased in Aged Mice

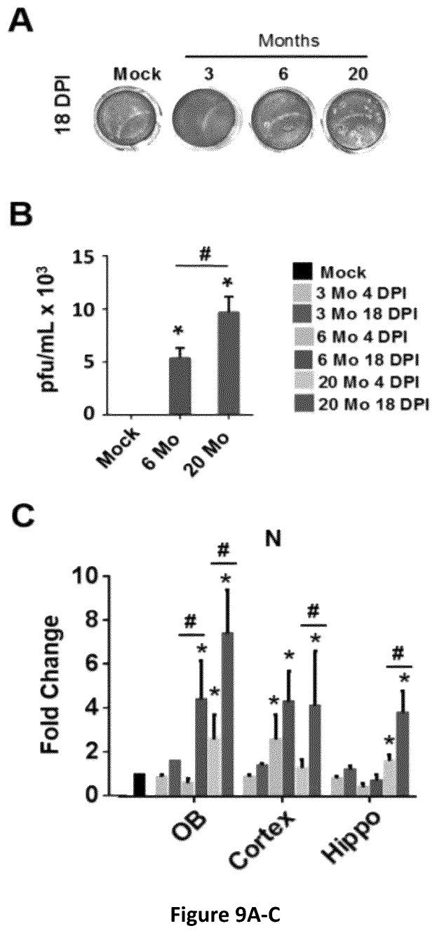

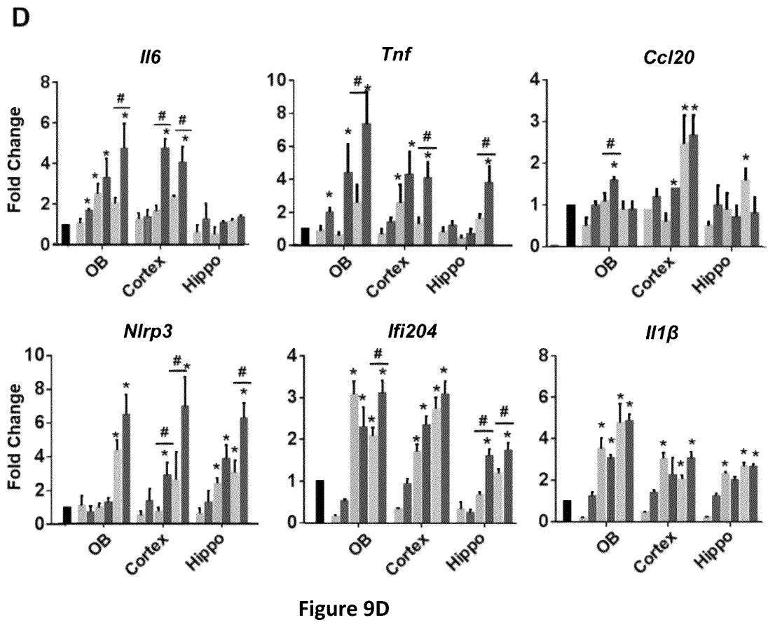

Since age is one of the crucial risk factors for the severity of infection, the inventors investigated the neurological consequences associated with CoV-2 infection in both young and old mice at both acute (4 days post-infection [DPI]) and post-acute (18 DPI) time points. Thus, 3-, 6-, and 20-month-old C57BL/6 mice were infected with 1×10⁵ of MA-10 intranasally. The infected mice were sacrificed 4 and 18 DPI, and viral titer and gene expression analyses were performed. A plaque assay was performed from the brain samples to quantitate the viral titer (FIGS. 9A and 9B). No plaques were detected in the infected young mice (3 months) or after mock infection at any age, but a significant number of plaques were detected in the infected adult (6 months) and in aged mice (20 months) groups with an age-dependent increase in viral titer and presence of N protein in the brains of infected mice. Also, the plaques were detected at only 18 DPI, and no plaques were detected in 4 DPI brain samples. qPCR analysis of RNA isolated from three brain regions (olfactory bulb, cortex, and hippocampus) confirmed the presence of CoV-2 N-protein transcript (FIG. 9C). Samples from 3-month-old mice did not contain any transcript. Six-month-old mice contained transcript in the cortex at 4 DPI and the cortex plus olfactory bulb at 18 DPI. Only the 20-month-old mice contained CoV-2 transcript in all brain regions at 18 DPI, additionally testing positive in the olfactory bulb and hippocampus at 4 DPI.

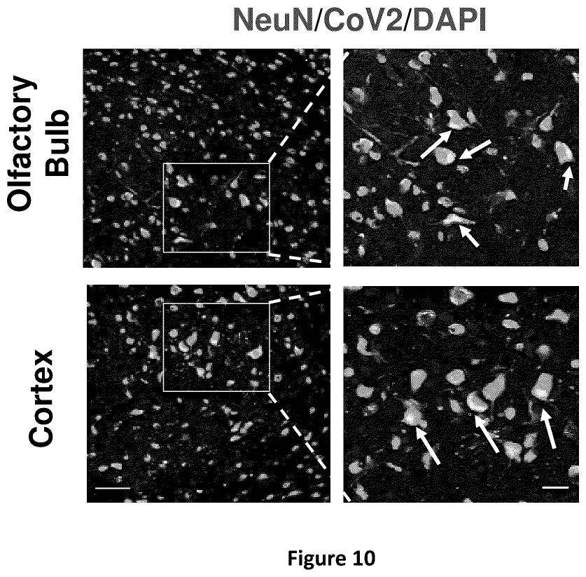

These results suggest that CoV2 neurotropism is correlated with correlated with advanced age. Presence of viral RNA in aged mouse brain was further confirmed in a separate experiment using fluorescent in situ hybridization (FISH) probe, as described in Rensen et al.,⁸³ herein incorporated in its entirety by reference. The inventors used 30 validated CoV2 specific nucleic acid primary probes to detect both the positive and negative strands of viral RNA to verify the extent of viral replication within each cell. In addition to the unique CoV2 specific region, each primary probe also contained an identical “FLAP” sequence that is complementary to a secondary probe conjugated with Cy3 for visualization by fluorescent microscopy. These oligos are hybridized to each other prior to their incubation with sample/tissue where they subsequently hybridize with CoV2 RNA. Using this FISH protocol showed evidence of CoV2 RNA colocalized to neurons (FIG. 10) in olfactory bulb and cortex.

### Neuroinflammation is Increased in Old Versus Young MA10-Infected Mice

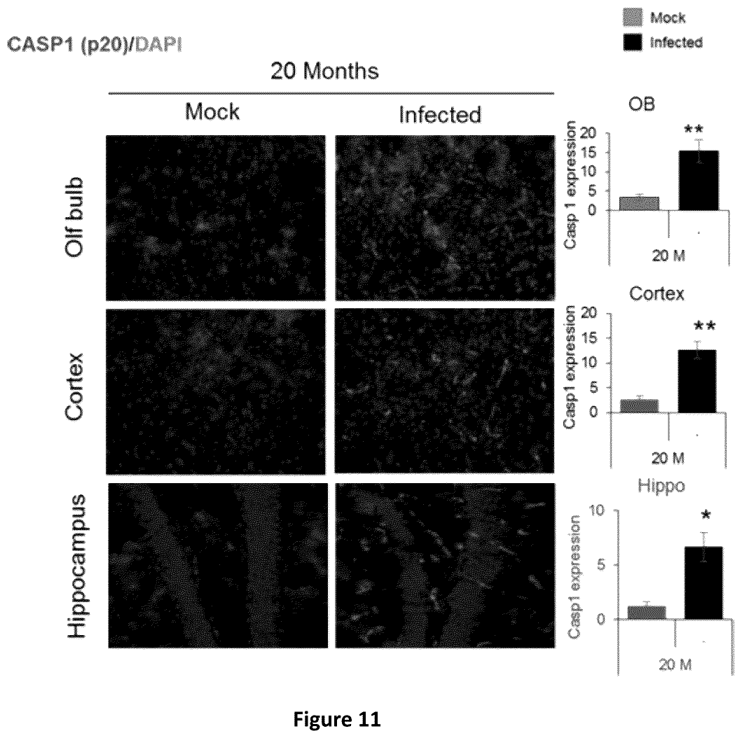

A qPCR analysis showed that the pro-inflammatory cytokines Il-6, Tnf-α, and Ccl20 were all generally increased in the 6- and 20-month-old infected mice, with the highest expression observed in the 20-month-old mice at 18 DPI (FIG. 9D). Only Il-6 and Tnf-α were significantly increased in the 3-month-old mice, and this increase was only observed in the olfactory bulb. The inflammasome component encoding genes Nlrp3 and IFI-16 (Ifi204 mouse homolog) were also significantly upregulated in 6- and 20-month-old mice along with Il-18 but were unchanged in 3-month-old mice. An increase in active caspase-1 (p20) was observed in all brain regions of the 20-month-old infected mice (FIG. 11). Overall, these data indicate that the intensity and pervasiveness of inflammation caused by CoV-2 infection in the brain increased with age and that inflammation may persist for at least 18 days post-infection.

### Expression of ADRD Risk/Pathology Genes is Increased in Aged MA10-Infected Mice

The inventors next examined the expression of five key AD risk genes identified by the network analysis in FIG. 8 (FKBP5, IFITM3, IFI16, CR1, and C5AR1). FKBP5 is a polyfunctional chaperone protein known to be associated with cognitive abnormalities and dysregulation of calcium homeostasis in AD.⁶² IFITM3 is induced by the interferon-driven inflammatory response and is known to increase the activity of γ-secretase for Aβ production.⁶³ Similarly, the interferon-stimulated gene IFI16 has been identified as a possible driver of neuroinflammation, and synaptic loss, in the AD brain.⁶⁴ IFI16 is an inflammasome component that serves as a viral nucleic acid sensor and promotes production of inter-leukin-1b (IL-1β).⁶⁵ CR1 is the receptor for complement factors C3b and C4b, which are known to be hyperactivated in COVID-19.55 It is expressed on antigen-presenting cells including microglia as well as on neurons.

C5AR1 is a receptor for C5a expressed primarily on myeloid cells and has been used as a biomarker for AD.⁵⁴ None of these genes were found to be upregulated in 3-month-old mice; however, all of them were significantly upregulated in 6- and 20-month-old mice, although Ifitm3 expression was lower in the hippocampus than in the olfactory bulb and cortex (FIG. 12A). An increase in Fkbp5 protein expression was confirmed in all CoV-2-infected brain regions by immunohistochemistry (FIG. 12B).

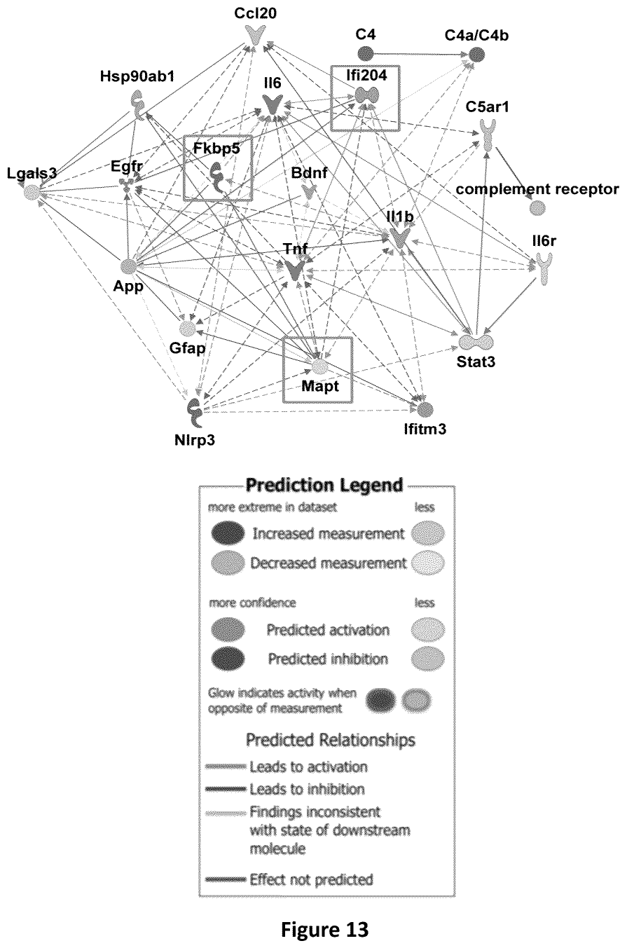

A similar increase in expression of inflammatory and ADRD risk genes to those shown in FIG. 8 was also recapitulated in 20-month-old mice (FIG. 13). Bioinformatic analyses of gene expression profiling data in our CoV2-MA10-vs mock-infected aged mouse brains showed evidence of viral neurotropism, prolonged viral infection, and changes in the expression of innate immune and proinflammatory genes. Further, qPCR analyses of fold change of differentially expressed genes in infected vs mock mice revealed upregulation of the proinflammatory genes (116, Tnfa, and Ccl20), and inflammasome genes (Ifi204, Nlrp3, Il1β), Then, Ingenuity Pathway Analysis (IPA) of the differentially expressed genes led to the identification of two ‘hub genes’, Fkbp5 and Ifi204, that are linked to Mapt expression, which is key to tauopathy (FIG. 13). Fkbp5 is a stabilizer of neurotoxic tau oligomers that promotes neuronal vulnerability in tauopathy and AD⁸⁴⁻⁸⁵. It is involved in tauopathy and is a member of the immunophilin protein family, which plays a role in immunoregulation and basic cellular processes involving protein folding and trafficking. It also interacts functionally with mature corticoid receptor hetero-complexes along with the 90-kDa heat shock and P23 proteins⁸⁶. Ifi204 is an interferon-inducible gene that acts as an important sensor of foreign nucleic acids and activators of the inflammasome response⁸⁷ which was reported to interact with the APOE*E2 allele as a modifier for the AD age-of-onset⁸⁸.

As noted above, both FKBP5 and IFI16 (human ortholog of Ifi204) genes were identified as ‘hub genes’ in the analyses of the whole transcriptome expression profile of the frontal cortex of COVID-19 and AD patients. These data, taken together, show that FKBP51 and IFI204 can serve as the lead candidate targets against CoV2-induced tauopathies and can be used to develop and test novel mechanisms underlying tauopathy.

Inflammation and ADRD Gene Signature are Correlated with Tau Pathology, α-Synuclein, and Demyelination

The inventors analyzed pooled CoV-2- or mock-infected mouse cortex samples for the expression of gene transcripts of additional AD risk genes by Nanostring nCounter Glial Profiling panel and Neuroinflammatory panel. These panels include key processes and pathways regulated under diseased conditions, such as cell stress and damage response (134 genes), glial regulation (180 genes), inflammation, and peripheral immune invasion (188 genes). The IPA analysis of Nanostring data (MA10-infected mouse brain samples) also showed a similar signature as that of the data collected from the GEO data-base (human brain tissue from CoV-2-infected patients). Upregulated genes include Lgals3, Egfr, C4a, Fkbp5, Gfap, Mapt, Ifitm3, Stat3, C5ar1, and Il6r. Downregulated genes include Hsp90ab1, Bdnf, and App (FIG. 12C).

The data from the inventors as well as others have suggested that CoV-2 infection may promote aberrant tau accumulation.⁶⁶ The inventors examined the tau pathology in MA10-infected mice by immunohistochemical staining using pT231 tau, an AD-relevant tau phosphorylation.⁶⁷ Brains from each group were sectioned and stained at 18 DPI. p-tau-positive cells were not present in the mock-infected mice or in the 3-month-old MA10-infected mice (FIG. 14A). However, the number of p-tau-positive cells was significantly elevated in all examined brain regions of the 6- and 20-month-old mice, with significant increases in the olfactory bulb and cortex of the 20-month-old group compared with the 6-month-old group.

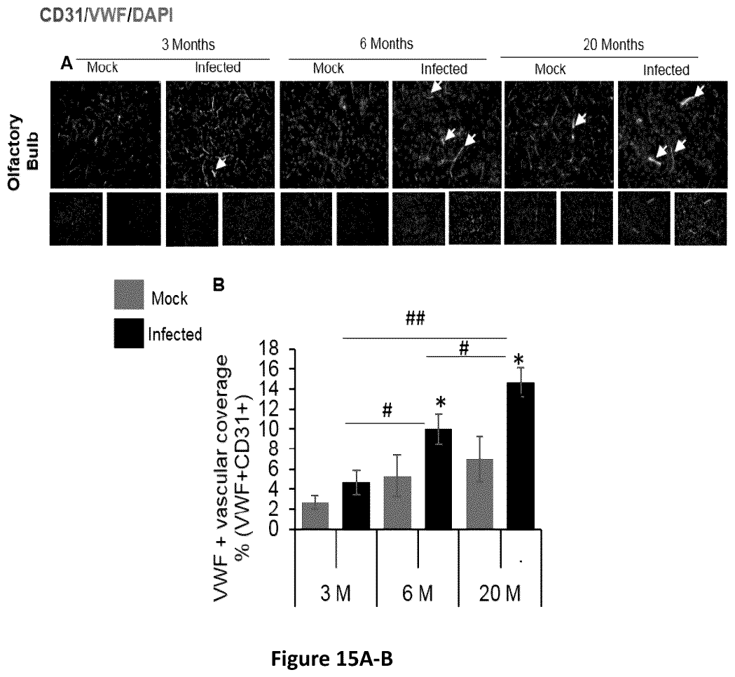

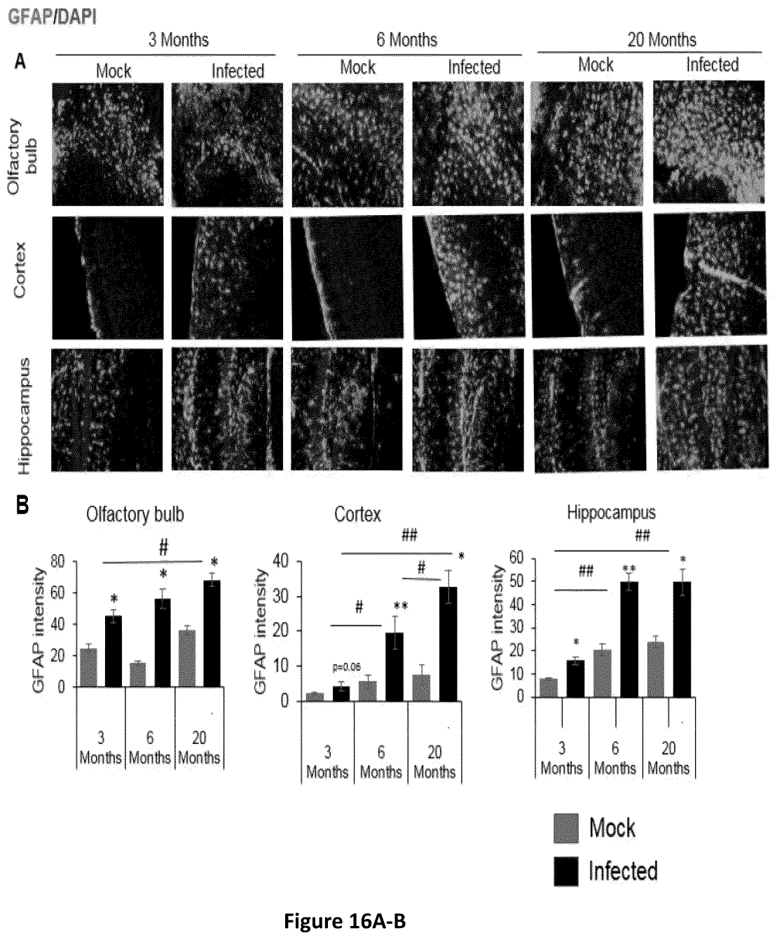

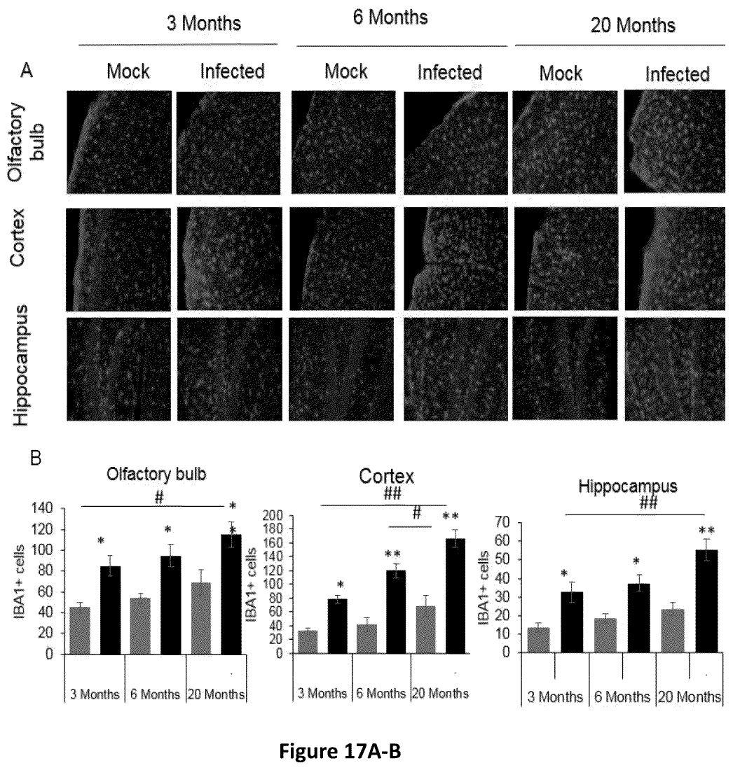

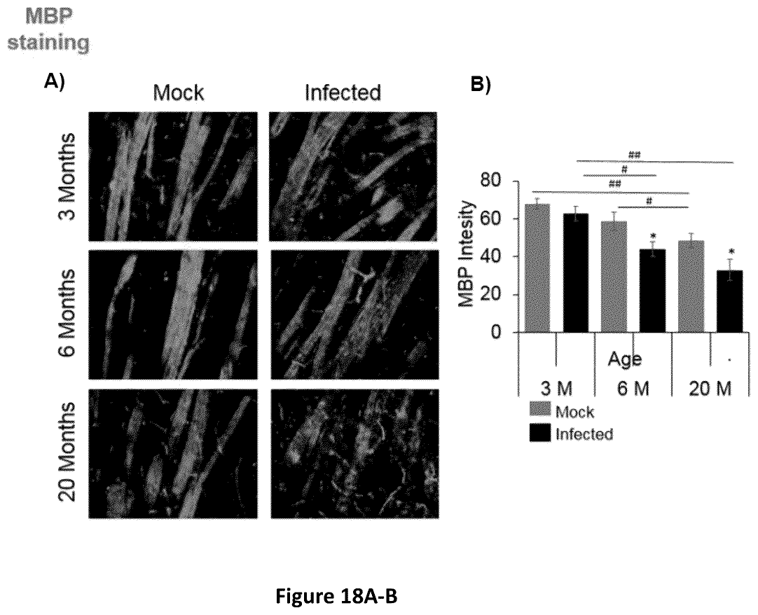

The inventors also examined the accumulation of tau oligomers, which data support as a toxic form of tau,⁶⁸⁻⁷⁰ using an oligomeric tau-specific antibody and again found a significant increase in the 6- and 20-month-old MA10-infected groups compared with mock as well as a significant increase in the 20-month-old MA10 group compared with the 6-month-old MA10 group in all brain regions (FIG. 14B). Similar increases in oligo-tau and p-tau were also recapitulated in 17-month-old mice (data not shown). Furthermore, p-tau was observed specifically in brain regions adjacent to CD31+ endothelial cells (FIG. 14C), which, in prior studies, was linked with blood-brain barrier dysfunction.⁷¹ Increased von Willebrand factor (vWF) staining in infected brains was observed as evidence of this blood-brain barrier (BBB) damage (FIG. 15). In addition, immunohistochemical analysis of CoV-2-infected brain tissue shows elevated GFAP expression (astroglial activation) and IBA1 expression (microglial activation) in the olfactory bulb, cortex, and hippocampus (FIGS. 16 and 17). A significantly increased grade of demyelination (MBP staining) in the striatum of infected aged brain tissues was observed along with significantly increased α-synuclein staining in the 6- and 20-month-old infected mice (FIGS. 18 and 19).

### Expression of IFI204, FKBP51 and Circulating Tau Proteins is Increased in CoV2-MA10 Infected Aged Mice.

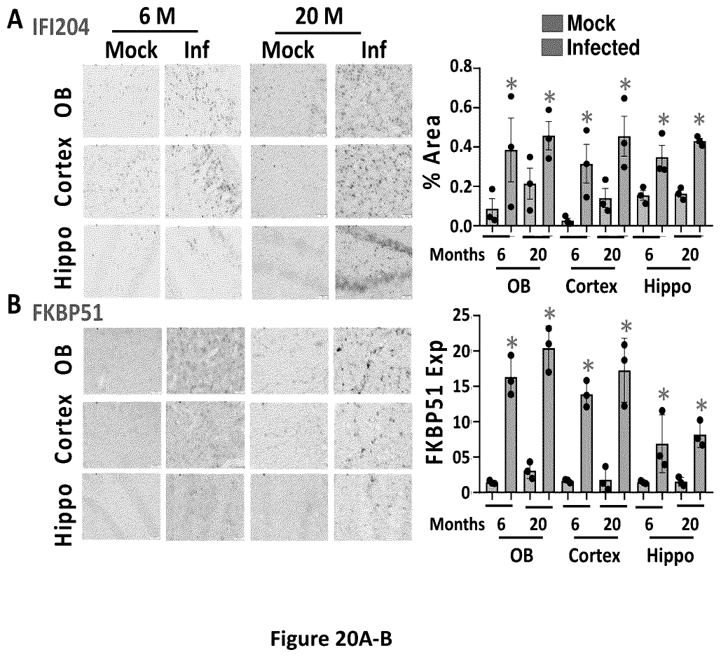

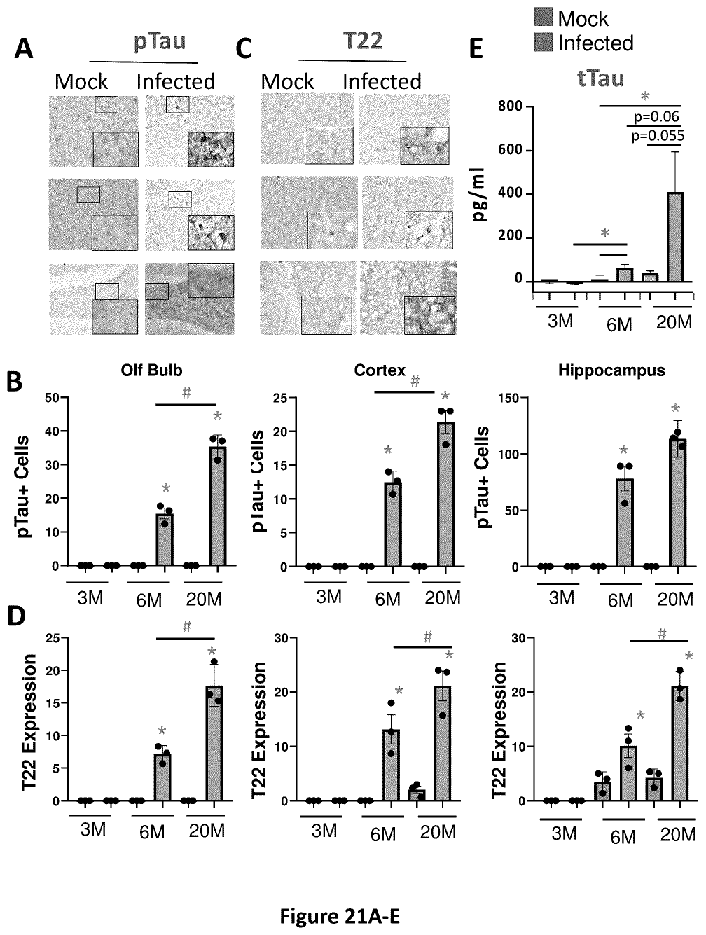

An increase in IFI204 and FKBP51 protein expression was confirmed in all three CoV2 infected brain regions by immunohistochemistry (IHC) (FIG. 20A-B). The microglial ionizing calcium-binding adaptor molecule 1 (IBA1) protein and astroglial GFAP were also found to be upregulated in an age-dependent manner. This increase in neuroinflammation was associated with a concomitant age-dependent increase in phosphorylation of tau (p-Tau) (FIG. 21A-B) and accumulation tau oligomers (FIG. 21C-D). More recently, the inventors measured circulating total tau (t-Tau) in serum of mock and infected mice using ELISA (Aβclonal) (FIG. 21E). No significant change was seen in 3-month-old mice (vs mock); however, t-Tau was significantly upregulated in 6- and 20-month-old mice after CoV2-MA10 infection with the highest upregulation seen in the 20-month-old mice.

### CoV2-MA10 Infection in PS19 Tau Transgenic Mice Accelerates Tau Pathology

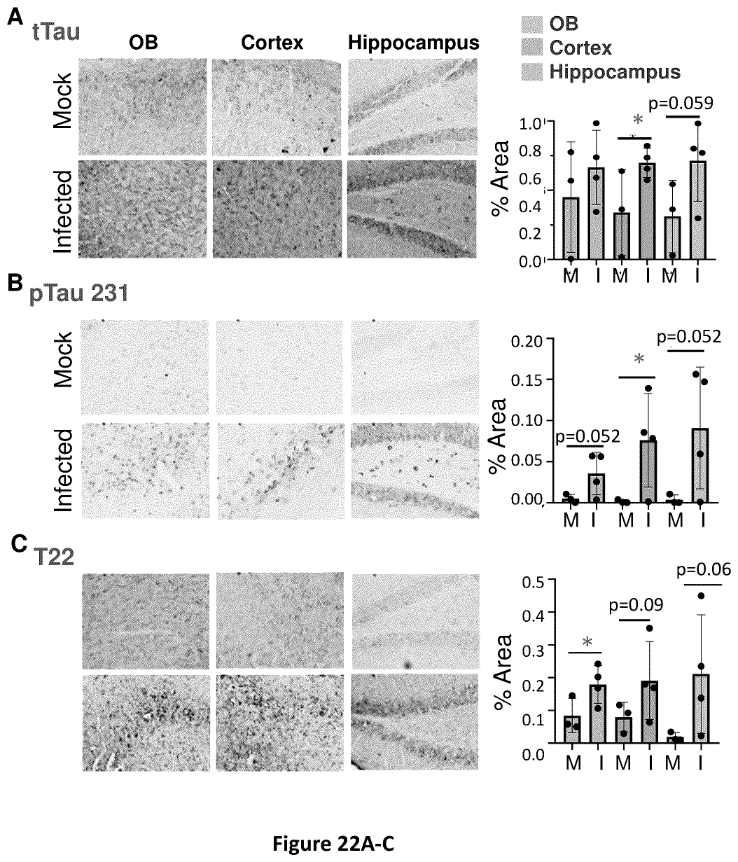

The PS19 tau transgenic mouse model overexpresses human 4R1N tau with the frontotemporal dementia-related P301S mutation driven by mouse prion protein promoter⁸⁹. This well-characterized and widely used tauopathy model has a moderate amount of tau accumulation by 6 months of age. The condition is progressive and is accompanied by neuronal loss in the hippocampus and cortex, gliosis, and cognitive impairments⁸⁹⁻⁹⁰. To examine how CoV2 infection affects tauopathy in PS19 mice, in a pilot study P301S (PS19) (male, 7-8 months-old) mice were infected IN with CoV-2 MA10 (1.25×10⁵ pfu) or mock (UV-inactivated) virus. Each day, following infection, the mice were individually examined for the signs of infection including changes in body temp, body weight respiration, and lethargy. The results at 18-day post-infection demonstrate accelerated tau accumulation in PS19 mice (FIG. 22), as seen by elevated levels of total (t-Tau), phosphorylated (pT231), and T22-positive oligomeric tau species in infected PS19 mice.

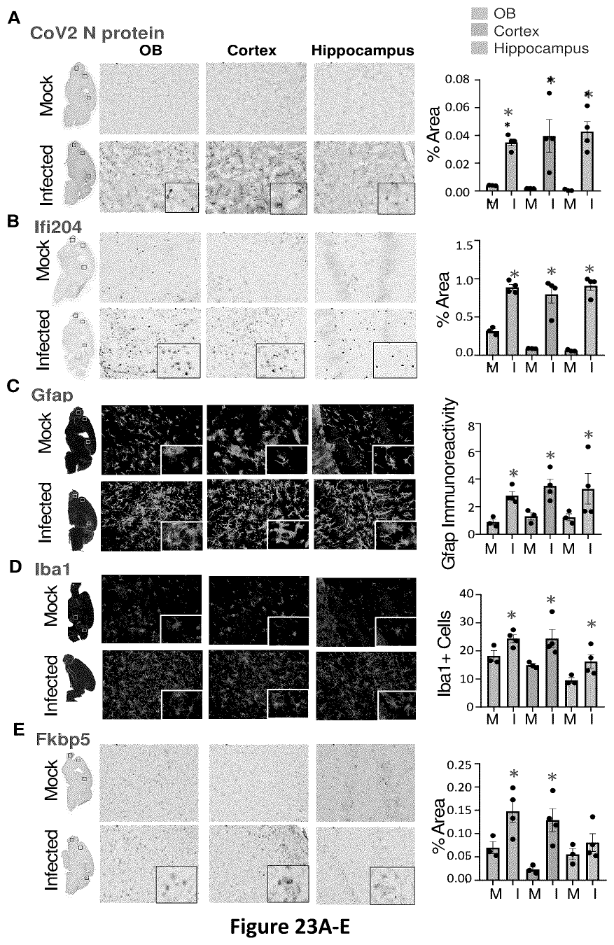

The inventors also found a significant upregulation of SARS-CoV-2 N protein (FIG. 23A) and concomitant upregulation of IFI204 protein in the OB, cortex, and hippocampus of CoV2-MA10 infected mice (FIG. 23B). In addition, there was an increase in levels of markers of neuroinflammation, GFAP and IBA1, observed in the OB, cortex, and hippocampus of CoV-2-MA10 infected mice (FIG. 23C, D). Finally, FKBP51 expression was significantly upregulated in the OB and cortex of infected mice, while trending levels were measured in the hippocampus of infected PS19 mice (FIG. 23E). These data suggest that CoV2-MA10 infection-induced neuroinflammation can accelerate the tau pathology in PS19 transgenic mice.

### FKBP51 Downregulation Reduced Tauopathy in CoV2-MA10 Infected Mice

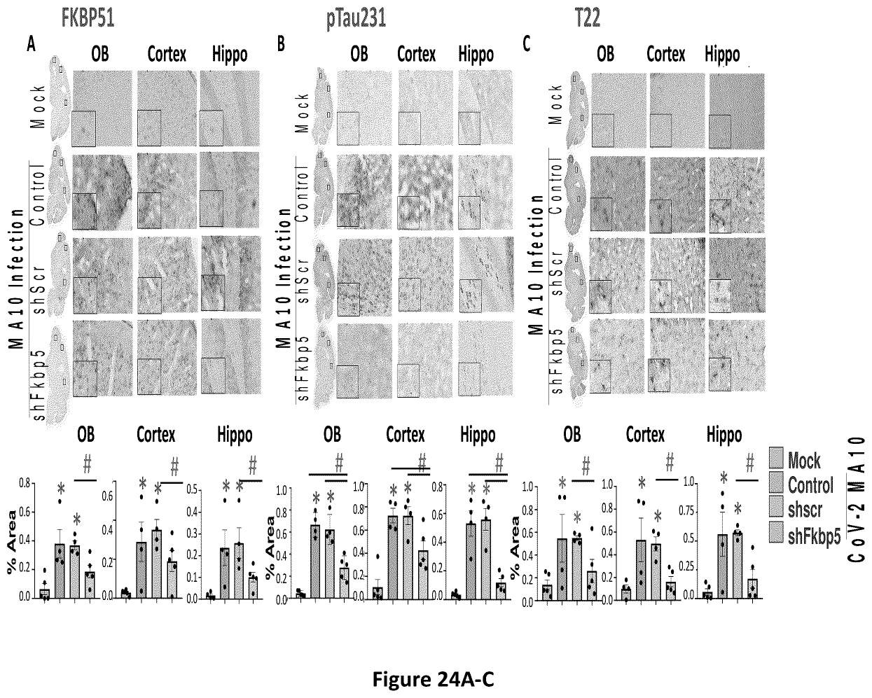

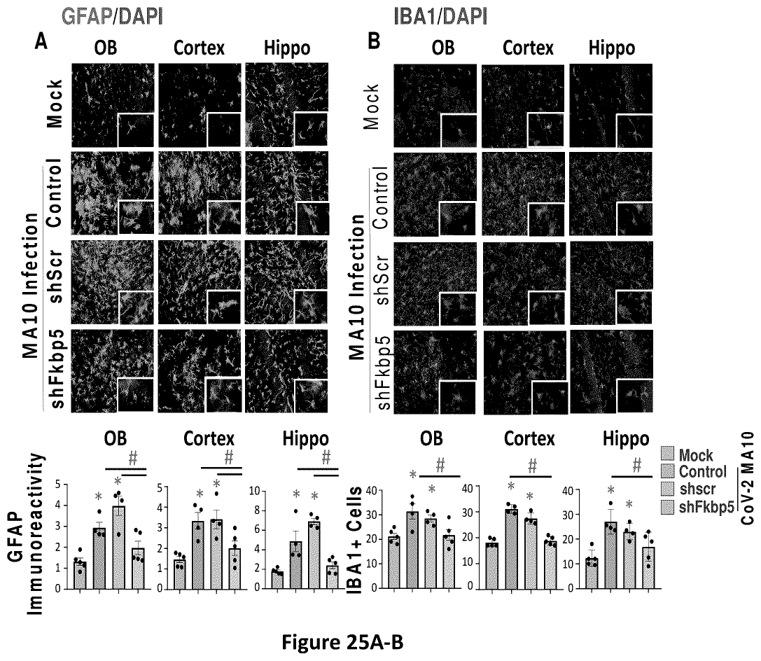

Since CoV2-MA10 infection significantly upregulated Fkbp5/FKBP51, the inventors reasoned that downregulation of Fkbp5 expression in infected mice can reduce CoV2-induced tauopathy. For in vivo downregulation of Fkbp5, the inventors used previously established PAMAM dendrimer based DPX technology⁹¹ described in Mayilsamy et al., herein incorporated by reference into this disclosure in its entirety, to deliver plasmid expressing small hairpin RNA (pshRNA) for Fkbp5. Briefly, two shRNA target sequences for Fkbp5 and scrambled sequences (as control) were cloned into pRP[2miR30]-Neo-CMV vector. C57Bl/6J (6-month-old) male were infected (IN) with 1.25×10⁵ pfu CoV-2 MA10 or mock (UV-inactivated) MA10 virus. Fkbp5 or scramble (Scr) targeting plasmids (20 μg) were complexed with extruded PAMAM dendrimers and DPXs were administered (IN) starting 24 hrs after infection. DPXs were delivered 3 times/wk for 2 wks. The mice were euthanized 18 dpi and brain sections examined by IHC. MA10 infection increased FKBP51, Tau phosphorylation and Tau oligomer accumulation in OB, cortex and hippocampus. IN treatment of pshFkbp5 significantly reduced FKBP51 protein expression compared to pshScramble in MA10 infected mice (FIG. 24A), however, it had no impact on infection burden or IFI204 expression (data not shown). On the other hand, reduction in FKBP51 protein was found associated with concomitant reductions in tau phosphorylation (FIG. 24B), tau oligomer accumulation (FIG. 24C) and neuroinflammation (IBA1 and GFAP expression) (FIG. 25) in CoV2-MA10 infected mice. Taken together, these results suggest that FKBP51 may serve as a potential mechanistic target for early onset tauopathy seen in adult CoV2-MA10 infected mice.

IFI204/NLRP3 Downregulation Reduced FKBP51 Expression in CoV2-infected HT22 Cells

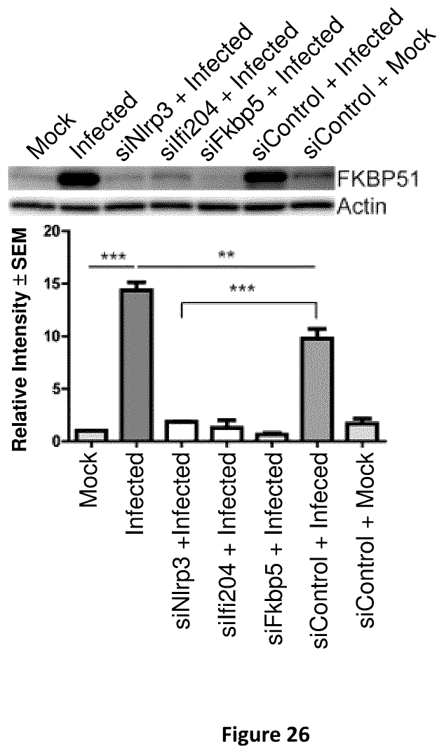

Since CoV2-MA10 infection induces FKBP51, IFI204 and NLRP3 as the lead targets due to their roles in tauopathy or inflammasome activation, the inventors examined whether knocking down IFI204 or NLRP3 expression will impact FKBP51. To test the role of these genes, a pool of three siRNAs for Fkbp51, Ifi204 and Nlrp3 (Dharmacon) were utilized. A universal scramble siRNA pool was used as a control. HT22 mouse neuronal cells were transfected with the siRNAs and, after 24 h of transfection, cells were infected with CoV2-MA10 virus. After 48 h post-infection, cells were harvested, and RNA examined for the expression of viral N gene expression by qPCR and cell lysates examined for FKBP51 expression. The results show that the cells transfected with siFkbp5, siNlrp3 or silfi204 all showed significant reduction of FKBP51 protein compared to scramble control. Also, compared to untransfected cells, the transfected cells showed some reduction of FKBP51 suggesting that the transfection reagent had some effect. While all treatments reduced FKBP51 expression compared to control siRNA (FIG. 26), none inhibited viral N gene expression (not shown).

### Materials and Methods

### Differential Gene Expression Analysis

Gene expression data from severe COVID-19 (n=12) or COVID-19-unaffected (n=12) individuals were obtained from the NCBI GEO database (GEO: GSE188847). In the associated study, frontal cortex tissues were collected within a post-mortem interval of less than 48-h. Unaffected patients were selected to be age and sex matched with COVID-19 patients. Salmon transcript quantification files for each patient were retrieved from GEO. A transcript to gene map file was exported from Ensembel Bio-Mart using GRCh38.p.13. Pooled differential gene analysis (12 COVID-19 versus 12 control samples) was performed using DESeq 2. The fold change output for each gene was filtered to only include significant changes (p % 0.05). Gene expression data from frontal cortex samples from patients with AD (n=40) and normal controls (n=22) were obtained from the GEO database (GEO: GSE118553). The GEO2R web tool (NCBI) was used to compare gene expression between AD and control groups, generating a list of differentially expressed genes with fold change and adjusted⁸² p value.

### Protein-Protein Network and Hub Gene Analysis

The lists of significantly differentially expressed genes in AD and COVID-19 were uploaded into the STRING database, and networks of gene interactions were obtained. Local network clusters matching genes upregulated in COVID-19 were ranked for their fit to the STRING database according to enrichment score. This entire network was imported into Cytoscape for hub gene analysis using the Cyto-hubba tool. The top 20 genes in 4 ranked algorithms; maximal clique centrality (MCC), density of maximum neighborhood component (DMNC), maximum neighborhood component (MNC), and Degree were identified, and the intersections between the four algorithms where two or more algorithms agreed were considered as the hub genes. This analysis was repeated for the AD versus control dataset. AD-related gene clusters were determined by taking the list of genes from the AD “disease” category in the IPA database (1,016 genes) and importing them into STRING as a network. Markov cluster algorithm (MCL) clustering was performed on the network with an inflation parameter of 2, and the top 8 clusters were selected for analysis.

### IPA

Alzheimer's genes were selected from literature (see FIG. 1). IPA (Qiagen) was used to plot known connections between the genes creating a molecular network. RNA sequencing (RNA-seq) profiling of human frontal cortex in severe COVID-19 (n=12) or unaffected (n=12) patients was obtained from the NCBI GEO database (GEO: GSE188847). Pooled differential gene analysis of COVID-19 versus unaffected patients was performed using DESeq2. Significantly differentially expressed genes (p<0.05) were uploaded into IPA, and their fold change information was overlaid onto the Alzheimer's gene network.

The IPA (Qiagen) database was queried for all genes known to contribute to or be altered in AD. The sub-set of these genes differentially expressed in COVID-19 were selected. Significantly differentially expressed genes (p<0.05) were uploaded into STRING, and the STRING database of protein-protein interactions was used to generate an interaction network. This network was imported into Cytoscape, and hub genes were identified within the network using Cytohubba. The IPA database was queried for genes known to increase susceptibility for Alzheimer's and to play a role in the early development of Alzheimer's. These Alzheimer's genes were connected to the hub genes using known relationships in the IPA database.

SARS-CoV-2 MA10 Virus Stock Preparation and Titration with Plaque-Based Assays

All experiments utilizing replication-competent SARS-CoV-2 were performed in a biosafety level 3 (BSL-3) laboratory at the University of South Florida. All viral stocks were produced and isolated from supernatants of Vero-E6 cells expressing human ACE2 and TMPRSS2 (BEI Resources NR-54970), cultured in T175 flasks to a confluency of 80%-90%, and infected with an original passage 2 (P2) MA10, at an MOI of 0.05 for 4 h, in 8 mL serum-free OptiMEM. Media was then replaced with 20 mL DMEM media supplemented with 5% FBS and 1% penicillin-streptomycin antibiotic and incubated for 72 h until clear cytopathic effect (CPE) was present. MA10 was obtained from BEI Resources (BEI Resources NR-55329). Supernatants were harvested, cleared of cell debris by centrifugation (500×g, 10 min) and filtration (0.45 mm), mixed with 10% SPG buffer (ATCC #MD9692), aliquoted, and stored at −80° C. Viral titers were quantified by determining the number of individual plaque-forming units (PFUs) after 72 h of infection on confluent Vero-E6 ACE2 TMPRSS2 cells. In brief, 25 mL viral stock was added to 225 mL OptiMEM (a10⁻¹ dilution) and was subsequently serially diluted (10-fold) in serum-free medium and inoculated on 7.5×10⁵ Vero E6 ACE2 TMPRSS2 cells in triplicates in a 48-well plate for 3 h. The inoculum was then removed and replaced with 0.8% carboxymethylcellulose in OptiMEM +2% FBS+1% penicillin-streptomycin antibiotic as overlay media for 72 h. The plates were then fixed with 80% methanol in water overnight, rinsed with 1×PBS, and stained with 0.1% crystal violet solution in ethanol. Plaques were then counted and calculated as PFU/mL.

### Animal Experiments

All animal procedures were conducted in accordance with the NIH guidelines for the Care and Use of Laboratory Animals and approved by the Institutional Animal Care and Use Committee of the University of South Florida. 3-, 6- and, 20-month-old male C57BL/6J mice were housed in a BSL-3 animal facility on a 12 h light-12 h dark cycle with food and water available ad libitum. Mice of each age group were infected with 100,000 PFUs of MA10 or mock ultraviolet light-inactivated MA10 that was generated and titrated using the previously described method. Mice under deep isoflurane anesthesia were intra-nasally inoculated with 50 mL (25 mL per nostril) of virus diluted in OptiMEM media. Mice were observed after inoculation for regain of consciousness and lucidity. Each subsequent day proceeding viral inoculation, the mice were individually examined for signs of infection including body weight change and alterations in body temperature, respiration, and lethargy. Mice were sacrificed 4 and 18 DPI. The brains and lungs were harvested and processed for plaque assay, RNA isolation, and immunostaining. Brain and lung tissue from mice were longitudinally sectioned in half, with half of one lung or brain sample going for RNA processing and the other half for plaque assay, while the second, intact lung was fixed with formalin for sectioning.

Plaque Assay from Organ Samples

Half of a longitudinally section organ (˜214 mg brain tissue or ˜50 mg lung) was homogenized in 200 mL OptiMEM media containing 10% penicillin-streptomycin-amphotericin antibiotic. The homogenate was then cleared of cell debris by centrifugation (500×g, 10 min). Viral titers from infected organs were quantified by determining the number of individual PFUs after 72 h of infection on confluent Vero-E6 ACE2 TMPRSS2 cells. 25 mL supernatant from the organ homogenate was added to 225 mL OptiMEM (10⁻¹ dilution) and then serially diluted (10-fold) in serum-free medium and inoculated on 7.5×10⁵ Vero E6 ACE2 TMPRSS2 cells in triplicates in a 48-well plate for 3 h. The inoculum was then removed and replaced with 0.8% carboxymethylcellulose in OptiMEM+2% FBS+1% penicillin-streptomycin antibiotic as overlay media for 72 h. The plates were then fixed with 80% methanol overnight, rinsed with 1×PBS, and stained with 0.1% crystal violet solution in ethanol. Plaques were then counted and calculated as PFU/mL of homogenate.

### RNA Isolation and PCR

Total RNA was isolated using Trizol (Life Technologies). The extracted RNA was subjected to nCounter gene expression analysis (NanoString Technologies) according to the manufacturer's instructions. Also, RNA was treated with DNAse I (Invitrogen, cat. no. 18068) to remove the residual genomic DNA. 1 mg RNA was used to prepare cDNA using Maxima Enzyme 5× reaction mix (Thermo Fisher Scientific). Quantitative real-time PCR reaction was performed using the cDNA in CFX384 Touch™ Real-Time PCR detection system (Bio-Rad). The reaction mixture was set up to 5 mL containing 1 mL 5×qPCR master mix, 0.5 mL forward primer and reverse primer, 1 mL water, and 1 mL cDNA. The reaction was per-formed using the following program: 95° C. or 3 min, followed by 45 cycles of 95° C. for 10 s, 60° C. for 1 min, and 72° C. for 15 s. All experiments were run in triplicate for three individual experiments. Gene expression from each age group was normalized to its respective mock control.

### Immunofluorescence Staining

Slide-mounted 30 mm brain sections were heated with antigen retrieval solution (1:100; Vector Laboratories, Burlingame, CA, USA) for 45 min at 90° C., cooled, and washed with PBS. The sections were permeabilized, and non-specific antigens were blocked for 1 h with serum-blocking solution (10% host serum, 0.2% Triton in PBS). Next, sections were incubated with primary antibody solution (5% host serum, 0.1% Triton X-100 in PBS) overnight at 4° C. After washing with PBS, sections were incubated with fluorescent tagged secondary antibody, washed again, dried, and mounted with DAPI containing anti-fade mounting medium.

### Immunoperoxidase Staining

For immunoperoxidase staining, after heat antigen retrieval, sections were treated with 3% hydrogen peroxide in water for 20 min, incubated with serum-blocking solution, and incubated overnight at 4° C. with primary antibody. Following washing, sections were then sequentially incubated with biotinylated secondary antibody for 2 h at room temperature, avidin-biotin peroxidase (ABC, 1:100 Vector Laboratories) for 1 h at room temperature, and 3,3⁰-Diaminobenzidine (DAB) substrate solution (Vector Laboratories) for 5 min. Sections were washed, dried, and cover slipped with DPX mounting medium. Bright-field or fluorescence images were taken with an Olympus X71 microscope using appropriate filters.

### Image Analysis and Quantitation

All quantitation was performed using NIH ImageJ Software. For immunohistochemical analysis, images were acquired using an Olympus IX71 microscope controlled by DP70 manager software (Olympus America, Melville, NY, USA). The images were taken at the same exposure and digital gain settings for a given magnification to minimize the differential background. Three sagittal brain sections were stained with each antibody and 3 images (20×) per region per section were captured, and analysis was performed. The images were converted to grayscale before quantification. The grayscale images were adjusted to exclude noise pixels. The same settings were used for quantifying all images. Positive cells were counted for p-tau, T22 oligomeric tau, IBA1, and a-synuclein staining using ImageJ software. Similarly, integrated density/unit area (immunoreactivity) was measured for FKBP5, MBP, and GFAP staining using ImageJ software. The results were expressed as mean number of positive cells or mean area of immunoreactivity ±standard error of mean (SEM).

### Statistical Analysis

All data are presented as mean±SEM. Statistical significance was evaluated by ANOVA with Holm-Sidak test for multiple comparisons if not mentioned otherwise. A p value of less than 0.05 was considered statistically significant for all comparisons.

### Data Availability

All whole-transcriptome gene expression datasets analyzed during the current study are available in the NCBI Gene Expression Omnibus repository under the accession numbers GEO: GSE188847 and GSE118553.

### CONCLUSION

The inventors have established a mouse-adapted CoV2-MA10 model in aged mice for in vivo mechanistic evaluation. Use of the model has shown that Cov2-MA10 is neurotropic in aged mice with increased neuroinflammation in old versus young Cov2-MA10 infected mice. Further, increased expression of tauopathy risk/pathology genes was shown in CoV2-MA10 infected aged mice with a correlation between inflammation and tauopathy gene signature and tau pathology. Important gene regulation nodes for FKBP5/FKBP51, IFI204 and inflammasome pathways were shown. The inventors show evidence of Ifi204/Nlrp3 genes upregulating FKBP51 expression and propose the potential of DPX-shFkbp5 to modulate tauopathy in CoV2-MA10 infected mice. CoV2-MA10 infection was established in a tauopathy mouse model which also showed that CoV2-MA10 is neurotropic in PS19 mice with evidence of increased neuroinflammation, accelerated tau pathology and enhanced expression of key signature proteins, FKBP51 and IFI204 in CoV2-MA10 infected PS19 mice. The results provide mechanistic targets for early onset tauopathy.

### REFERENCES

1. Thies, W., and Bleiler, L.; Alzheimer's Association (2013). 2013 Alzheimer's disease facts and figures. Alzheimers Dement. 9, 208-245.
2. Knopman, D. S., Amieva, H., Petersen, R. C., Chételat, G., Holtzman, D. M., Hyman, B. T., Nixon, R. A., and Jones, D. T. (2021). Alzheimer disease. Nat. Rev. Dis. Primers 7, 33.
3. Nelson, P. T., Alafuzoff, I., Bigio, E. H., Bouras, C., Braak, H., Cairns, N. J., Castellani, R. J., Crain, B. J., Davies, P., Del Tredici, K., et al. (2012). Correlation of Alzheimer disease neuropathologic changes with cognitive status: a review of the literature. J. Neuropathol. Exp. Neurol. 71, 362-381.
4. Bejanin, A., Schonhaut, D. R., La Joie, R., Kramer, J. H., Baker, S. L., Sosa, N., Ayakta, N., Cantwell, A., Janabi, M., Lauriola, M., et al. (2017). Tau pathology and neurode-generation contribute to cognitive impairment in Alzheimer's disease. Brain 140, 3286-3300.
5. Xia, C., Makaretz, S. J., Caso, C., McGinnis, S., Gomperts, S. N., Sepulcre, J., Gomez-Isla, T., Hyman, B. T., Schultz, A., Vasdev, N., et al. (2017). Association of in vivo [18F] AV-1451 tau PET imaging results with cortical atrophy and symptoms in typical and atypical Alzheimer disease. JAMA Neurol. 74, 427-436.
6. Wainberg, M., Luquez, T., Koelle, D. M., Readhead, B., Johnston, C., Darvas, M., and Funk, C. C. (2021). The viral hypothesis: how herpesviruses may contribute to Alzheimer's disease. Mol. Psychiatr. 26, 5476-5480.
7. Sulzer, D., Antonini, A., Leta, V., Nordvig, A., Smeyne, R. J., Goldman, J. E., Al-Dalahmah, O., Zecca, L., Sette, A., Bubacco, L., et al. (2020). COVID-19 and possible links with Parkinson's disease and parkinsonism: from bench to bedside. NPJ Parkinsons Dis. 6, 18.
8. Tsai, J. P., and Baker, A. J. (2013). Influenza-associated neurological complications. Neurocrit. Care 18, 118-130.
9. Li, H., Liu, C. C., Zheng, H., and Huang, T. Y. (2018). Amyloid, tau, pathogen infection and antimicrobial protection in Alzheimer's disease-conformist, nonconformist, and realistic prospects for A D pathogenesis. Transl. Neurodegener. 7, 34.
10. Sy, M., Kitazawa, M., Medeiros, R., Whitman, L., Cheng, D., Lane, T. E., and Laferla, F. M. (2011). Inflammation induced by infection potentiates tau pathological features in transgenic mice. Am. J. Pathol. 178, 2811-2822.
11. Lee, D. C., Rizer, J., Selenica, M. L. B., Reid, P., Kraft, C., Johnson, A., Blair, L., Gordon, M. N., Dickey, C. A., and Morgan, D. (2010). LPS-induced inflammation exacerbates phospho-tau pathology in rTg4510 mice. J. Neuroinflammation 7, 56.
12. Ising, C., Venegas, C., Zhang, S., Scheiblich, H., Schmidt, S. V., Vieira-Saecker, A., Schwartz, S., Albasset, S., McManus, R. M., Tejera, D., et al. (2019). NLRP3 inflammasome activation drives tau pathology. Nature 575, 669-673.
13. Morales, I., Jiménez, J. M., Mancilla, M., and Maccioni, R. B. (2013). Tau oligomers and fibrils induce activation of microglial cells. J. Alzheimers Dis. 37, 849-856.
14. World Health Organization (2020). WHO COVID-19 dashboard.
15. Malkova, A., Kudryavtsev, I., Starshinova, A., Kudlay, D., Zinchenko, Y., Glushkova, A., Yablonskiy, P., and Shoenfeld, Y. (2021). Post COVID-19 syndrome in patients with asymptomatic/mild form. Pathogens 10, 1408.
16. Jiang, R. D., Liu, M. Q., Chen, Y., Shan, C., Zhou, Y. W., Shen, X. R., Li, Q., Zhang, L., Zhu, Y., Si, H. R., et al. (2020). Pathogenesis of SARS-CoV-2 in transgenic mice expressing human Angiotensin-converting Enzyme 2. Cell 182, 50-58.e8.
17. Mao, L., Jin, H., Wang, M., Hu, Y., Chen, S., He, Q., Chang, J., Hong, C., Zhou, Y., Wang, D., et al. (2020). Neurologic manifestations of hospitalized patients with Coronavirus disease 2019 in Wuhan, China. JAMA Neurol. 77, 683-690.
18. Mao, L., Wang, M., Chen, S., He, Q., Chang, J., Hong, C., Zhou, Y., Wang, D., Li, Y., Jin, H., and Hu, B. (2020). Neurological manifestations of hospitalized patients with COVID-19 in Wuhan, China: a retrospective case series study.
19. Ye, M., Ren, Y., and Lv, T. (2020). Encephalitis as a clinical manifestation of COVID-Brain Behav. Immun. 88, 945-946.
20. Kim, G. U., Kim, M. J., Ra, S. H., Lee, J., Bae, S., Jung, J., and Kim, S. H. (2020). Clinical characteristics of asymptomatic and symptomatic patients with mild COVID-19. Clin. Microbiol. Infect. 26, 948.e1-948.e3.
21. Beyrouti, R., Adams, M. E., Benjamin, L., Cohen, H., Farmer, S. F., Goh, Y. Y., Humphries, F., Jäger, H. R., Losseff, N. A., Perry, R. J., et al. (2020). Characteristics of ischaemic stroke associated with COVID-19. J. Neurol. Neurosurg. Psychiatry 91, 889-891.
22. Benussi, A., Pilotto, A., Premi, E., Libri, I., Giunta, M., Agosti, C., Alberici, A., Baldelli, E., Benini, M., Bonacina, S., et al. (2020). Clinical characteristics and outcomes of in-patients with neurologic disease and COVID-19 in Brescia, Lombardy, Italy. Neurology 95, e910-e920.
23. Poyiadji, N., Shahin, G., Noujaim, D., Stone, M., Patel, S., and Griffith, B. (2020). COVID-19-associated acute hemorrhagic necrotizing encephalopathy: C T and MRI features. Radiology 296, 201187.
24. Tape, C., Byrd, K. M., Aung, S., Lonks, J. R., Flanigan, T. P., and Rybak, N. R. (2020). COVID-19 in a patient presenting with syncope and a normal chest X-ray. R. I. Med. J. 103, 50-51.
25. Poyiadji, N., Shahin, G., Noujaim, D., Stone, M., Patel, S., and Griffith, B. (2020). COVID-19-associated acute hemorrhagic necrotizing encephalopathy: imaging features. Radiology 296, E119-E120.
26. von Weyhern, C. H., Kaufmann, I., Neff, F., and Kremer, M. (2020). Early evidence of pronounced brain involvement in fatal COVID-19 outcomes. Lancet 395, e109.
27. Douaud, G., Lee, S., Alfaro-Almagro, F., Arthofer, C., Wang, C., McCarthy, P., Lange, F., Andersson, J. L. R., Griffanti, L., Duff, E., et al. (2022). SARS-CoV-2 is associated with changes in brain structure in U K Biobank. Nature 604, 697-707.
28. Qin, Y., Wu, J., Chen, T., Li, J., Zhang, G., Wu, D., Zhou, Y., Zheng, N., Cai, A., Ning, Q., et al. (2021). Long-term microstructure and cerebral blood flow changes in patients-recovered from COVID-19 without neurological manifestations. J. Clin. Invest. 131, 147329.
29. Howell, M. C., Green, R., McGill, A. R., Kahlil, R. M., Dutta, R., Mohapatra, S. S., and Mohapatra, S. (2021). Activation of intracellular complement in lungs of patients with severe COVID-19 disease decreases T-cell activity in the lungs. Front. Immunol. 12, 700705.
30. McGill, A. R., Kahlil, R., Dutta, R., Green, R., Howell, M., Mohapatra, S., and Mohapatra, S. S. (2021). SARS-CoV-2 immuno-pathogenesis and potential for diverse vaccines and therapies: opportunities and challenges. Infect. Dis. Rep. 13, 102-125.
31. Xu, D., Biswal, M., Neal, A., and Hai, R. (2021). Review Devil's tools: SARS-CoV-2 antagonists against innate immunity. Curr. Res. Virol. Sci. 2, 100013.
32. Goldeck, D., Larbi, A., Pellicanó, M., Alam, I., Zerr, I., Schmidt, C., Fulop, T., and Pawelec, G. (2013). Enhanced chemokine receptor expression on leukocytes of patients with Alzheimer's disease. PLOS One 8, e66664.
33. Corces, M. R., Shcherbina, A., Kundu, S., Gloudemans, M. J., Frésard, L., Granja, J. M., Louie, B. H., Eulalio, T., Shams, S., Bagdatli, S. T., et al. (2020). Single-cell epigenomic analyses implicate candidate causal variants at inherited risk loci for Alzheimer's and Parkinson's diseases. Nat. Genet. 52, 1158-1168.
34. Novikova, G., Kapoor, M., Tcw, J., Abud, E. M., Efthymiou, A. G., Chen, S. X., Cheng, H., Fullard, J. F., Bendl, J., Liu, Y., et al. (2021). Integration of Alzheimer's disease genetics and myeloid genomics identifies disease risk regulatory elements and genes. Nat. Commun. 12, 1610.
35. Zuena, A. R., Casolini, P., Lattanzi, R., and Maftei, D. (2019). Chemokines in Alzheimer's disease: new insights into prokineticins, chemokine-like proteins. Front. Pharmacol. 10, 622.
36. Mansour, H. M., Fawzy, H. M., El-Khatib, A. S., and Khattab, M. M. (2022). Repurposed anti-cancer epidermal growth factor receptor inhibitors: mechanisms of neuroprotective effects in Alzheimer's disease. Neural Regen. Res. 17, 1913-1918.
37. Cicognola, C., Janelidze, S., Hertze, J., Zetterberg, H., Blennow, K., Mattsson-Carlgren, N., and Hansson, O. (2021). Plasma glial fibrillary acidic protein detects Alzheimer pathology and predicts future conversion to Alzheimer dementia in patients with mild cognitive impairment. Alzheimer's Res. Ther. 13, 68.
38. Vélez, J. I., Lopera, F., Sepulveda-Falla, D., Patel, H. R., Johar, A. S., Chuah, A., Tobón, C., Rivera, D., Villegas, A., Cai, Y., et al. (2016). APOE*E2 allele delays age of onset in PSEN1 E280A Alzheimer's disease. Mol. Psychiatr. 21, 916-924.
39. Brigas, H. C., Ribeiro, M., Coelho, J. E., Gomes, R., Gomez-Murcia, V., Carvalho, K., Faivre, E., Costa-Pereira, S., Darrigues, J., de Almeida, A. A., et al. (2021). IL-17 triggers the onset of cognitive and synaptic deficits in early stages of Alzheimer's disease. Cell Rep. 36, 109574.
40. Ojala, J. O., and Sutinen, E. M. (2017). The role of interleukin-18, oxidative stress and metabolic syndrome in Alzheimer's disease. J. Clin. Med. 6, E55.
41. Haddick, P. C. G., Larson, J. L., Rathore, N., Bhangale, T. R., Phung, Q. T., Srinivasan, K., Hansen, D. V., Lill, J. R.; Alzheimer's Disease Genetic Consortium ADGC Alzheimer's Disease Neuroimaging Initiative ADNI, and Pericak-Vance, M. A., et al. (2017). A common variant of IL-6R is associated with elevated IL-6 pathway activity in Alzheimer's disease brains. J. Alzheimers Dis. 56, 1037-1054.
42. Cheng, Z., Zou, X., Jin, Y., Gao, S., Lv, J., Li, B., and Cui, R. (2018). The role of KLF4 in Alzheimer's disease. Front. Cell. Neurosci. 12, 325.
43. Tan, Y., Zheng, Y., Xu, D., Sun, Z., Yang, H., and Yin, Q. (2021). Galectin-3: a key player in microglia-mediated neuroinflammation and Alzheimer's disease. Cell Biosci. 11, 78.
44. Tao, C. C., Cheng, K. M., Ma, Y. L., Hsu, W. L., Chen, Y. C., Fuh, J. L., Lee, W. J., Chao, C. C., and Lee, E. H. Y. (2020). Galectin-3 promotes Abeta oligomerization and Abeta toxicity in a mouse model of Alzheimer's disease. Cell Death Differ. 27, 192-209.
45. Dharshini, S. A. P., Taguchi, Y. H., and Gromiha, M. M. (2019). Investigating the energy crisis in Alzheimer disease using transcriptome study. Sci. Rep. 9, 18509.
46. Gaudreault, S. B., Dea, D., and Poirier, J. (2004). Increased caveolin-1 expression in Alzheimer's disease brain. Neurobiol. Aging 25, 753-759.
47. Wang, L., Pavlov, P. F., Kumar, R., and Winblad, B. (2020). FKBP51-Hsp90 complex as a novel therapeutic target for Alzheimer's disease. Alzheimers Dement. 16.
48. Fujii, T., Ota, M., Hori, H., Hattori, K., Teraishi, T., Matsuo, J., Kinoshita, Y., Ishida, I., Nagashima, A., and Kunugi, H. (2014). The common functional FKBP5 variant rs1360780 is associated with altered cognitive function in aged individuals. Sci. Rep. 4, 6696.
49. Blair, L. J., Nordhues, B. A., Hill, S. E., Scaglione, K. M., O'Leary, J. C., 3rd, Fontaine, S. N., Breydo, L., Zhang, B., Li, P., Wang, L., et al. (2013). Accelerated neurodegeneration through chaperone-mediated oligomerization of tau. J. Clin. Invest. 123, 4158-4169.
50. Blair, L. J., Sabbagh, J. J., and Dickey, C. A. (2014). Targeting Hsp90 and its co-chaperones to treat Alzheimer's disease. Expert Opin. Ther. Targets 18, 1219-1232.
51. Loeffler, D. A., Klaver, A. C., Coffey, M. P., Aasly, J. O., and LeWitt, P. A. (2016). Agerelated decrease in heat shock 70-kDa protein 8 in cerebrospinal fluid is associated with increased oxidative stress. Front. Aging Neurosci. 8, 178.
52. Hur, J. Y. (2021). Innate immunity protein IFITM3 in Alzheimer's disease. DNA Cell Biol. 40, 1351-1355.
53. Tenner, A. J. (2020). Complement-mediated events in Alzheimer's disease: mechanisms and potential therapeutic targets. J. Immunol. 204, 306-315.
54. Hernandez, M. X., Jiang, S., Cole, T. A., Chu, S. H., Fonseca, M. I., Fang, M. J., Hohsfield, L. A., Torres, M. D., Green, K. N., Wetsel, R. A., et al. (2017). Prevention of C5aR1 signaling delays microglial inflammatory polarization, favors clearance pathways and suppresses cognitive loss. Mol. Neurodegener. 12, 66.
55. Zhu, X. C., Yu, J. T., Jiang, T., Wang, P., Cao, L., and Tan, L. (2015). CR1 in Alzheimer's disease. Mol. Neurobiol. 51, 753-765.
56. Sanfilippo, C., Castrogiovanni, P., Imbesi, R., and Di Rosa, M. (2020). CHI3L2 expression levels are correlated with AIF1, PECAM1, and CALB1 in the brains of Alzheimer's disease patients. J. Mol. Neurosci. 70, 1598-1610.
57. Sabbir, M. G. (2018). Loss of Ca (2+)/calmodulin dependent protein kinase kinase 2 leads to aberrant transferrin phosphorylation and trafficking: a potential biomarker for Alzheimer's disease. Front. Mol. Biosci. 5, 99.
58. Mairet-Coello, G., Courchet, J., Pieraut, S., Courchet, V., Maximov, A., and Polleux, F. (2013). The CAMKK2-AMPK kinase pathway mediates the synaptotoxic effects of Abeta oligomers through Tau phosphorylation. Neuron 78, 94-108.
59. Elliott, E., Atlas, R., Lange, A., and Ginzburg, I. (2005). Brain-derived neurotrophic factor induces a rapid dephosphorylation of tau protein through a PI-3 Kinase signaling mechanism. Eur. J. Neurosci. 22, 1081-1089.
60. Plagman, A., Hoscheidt, S., McLimans, K. E., Klinedinst, B., Pappas, C., Anantharam, V., Kanthasamy, A., and Willette, A. A.; Alzheimer's Disease Neuroimaging Initiative (2019). Cholecystokinin and Alzheimer's disease neuroimaging. Neurobiol. Aging 76, 201-207.
61. Shibata, N., Kawarai, T., Meng, Y., Lee, J. H., Lee, H. S., Wakutani, Y., Shibata, E., Pathan, N., Bi, A., Sato, C., et al. (2007). Association studies between the plasmin genes and late-onset Alzheimer's disease. Neurobiol. Aging 28, 1041-1043.
62. Cao, W., and Konsolaki, M. (2011). FKBP immunophilins and Alzheimer's disease: a chaperoned affair. J. Biosci. 36, 493-498.
63. Hur, J. Y., Frost, G. R., Wu, X., Crump, C., Pan, S. J., Wong, E., Barros, M., Li, T., Nie, P., Zhai, Y., et al. (2020). The innate immunity protein IFITM3 modulates gamma-secretase in Alzheimer's disease. Nature 586, 735-740.
64. Roy, E. R., Wang, B., Wan, Y. W., Chiu, G., Cole, A., Yin, Z., Propson, N. E., Xu, Y., Jankowsky, J. L., Liu, Z., et al. (2020). Type I interferon response drives neuroinflammation and synapse loss in Alzheimer disease. J. Clin. Invest. 130, 1912-1930.
65. Coulon, P. G., Dhanushkodi, N., Prakash, S., Srivastava, R., Roy, S., Alomari, N. I., Nguyen, A. M., Warsi, W. R., Ye, C., Carlos-Cruz, E. A., et al. (2019). NLRP3, NLRP12, and IFI16 inflammasomes induction and caspase-1 activation triggered by virulent HSV-1 strains are associated with severe corneal inflammatory herpetic disease. Front. Immunol. 10, 1631.
66. Reiken, S., Sittenfeld, L., Dridi, H., Liu, Y., Liu, X., and Marks, A. R. (2022). Alzheimer's-like signaling in brains of COVID-19 patients. Alzheimers Dement. 18, 955-965.
67. Luna-Viramontes, N. I., Campa-Córdoba, B. B., Ontiveros-Torres, M. A., Harrington, C. R., Villanueva-Fierro, I., Guadarrama-Ortíz, P., Garcés-Ramírez, L., de la Cruz, F., Hernandes-Alejandro, M., Martínez-Robles, S., et al. (2020). PHF-core tau as the potential initiating event for tau pathology in Alzheimer's disease. Front. Cell. Neurosci. 14, 247.
68. Ward, S. M., Himmelstein, D. S., Lancia, J. K., and Binder, L. I. (2012). Tau oligomers and tau toxicity in neurodegenerative disease. Biochem. Soc. Trans. 40, 667-671.
69. Martinisi, A., Flach, M., Sprenger, F., Frank, S., Tolnay, M., and Winkler, D. T. (2021). Severe oligomeric tau toxicity can be reversed without long-term sequelae. Brain 144, 963-974.
70. Shafiei, S. S., Guerrero-Muñoz, M. J., and Castillo-Carranza, D. L. (2017). Tau oligomers: cytotoxicity, propagation, and mitochondrial damage. Front. Aging Neurosci. 9, 83.
71. Blair, L. J., Frauen, H. D., Zhang, B., Nordhues, B. A., Bijan, S., Lin, Y. C., Zamudio, F., Hernandez, L. D., Sabbagh, J. J., Selenica, M. L. B., and Dickey, C. A. (2015). Tau depletion prevents progressive blood-brain barrier damage in a mouse model of tauopathy. Acta Neuropathol. Commun. 3, 8.
72. Wang, F., Xu, J., Xu, S. J., Guo, J. J., Wang, F., and Wang, Q. W. (2022). Analysis and identification genetic effect of SARS-CoV-2 infections to Alzheimer's disease patients by integrated bioinformatics. J. Alzheimers Dis. 85, 729-744.
73. Das, M., Penn, C., Martinez, T., Mayilsamy, K., McGill, A., Wiling, A., Mohapatra, S. S., and Mohapatra, S. (2020). COVID-19 neurotropism and implications for therapy. Neuroimmunol. Neuroinflamm. 7, 141-149.
74. Rutkai, I., Mayer, M. G., Hellmers, L. M., Ning, B., Huang, Z., Monjure, C. J., Coyne, C., Silvestri, R., Golden, N., Hensley, K., et al. (2022). Neuropathology and virus in brain of SARS-CoV-2 infected non-human primates. Nat. Commun. 13, 1745.
75. Palomer, E., Buechler, J., and Salinas, P. C. (2019). Wnt signaling deregulation in the aging and Alzheimer's brain. Front. Cell. Neurosci. 13, 227.
76. Seaks, C. E., and Wilcock, D. M. (2020). Infectious hypothesis of Alzheimer disease. PLOS Pathog. 16, e1008596.
77. Abbott, A. (2020). Are infections seeding some cases of Alzheimer's disease? Nature 587, 22-25.
78. Twohig, D., and Nielsen, H. M. (2019). alpha-synuclein in the pathophysiology of Alzheimer's disease. Mol. Neurodegener. 14, 23.
79. Haass, C., Kaether, C., Thinakaran, G., and Sisodia, S. (2012). Trafficking and proteolytic processing of APP. Cold Spring Harb. Perspect. Med. 2, a006270.
80. Winslow, A. R., Moussaud, S., Zhu, L., Post, K. L., Post, K. L., Dickson, D. W., Berezovska, O., and McLean, P. J. (2014). Convergence of pathology in dementia with Lewy bodies and Alzheimer's disease: a role for the novel interaction of alpha-synuclein and presenilin 1 in disease. Brain 137, 1958-1970.
81. Kaneko, H., Kakita, A., Kasuga, K., Nozaki, H., Ishikawa, A., Miyashita, A., Kuwano, R., Ito, G., Iwatsubo, T., Takahashi, H., et al. (2007). Enhanced accumulation of phosphorylated alpha-synuclein and elevated beta-amyloid 42/40 ratio caused by expression of the presenilin-1 deltaT440 mutant associated with familial Lewy body disease and variant Alzheimer's disease. J. Neurosci. 27, 13092-13097.
82. Benjamini, Y., and Hochberg, Y. (1995). Controlling the false discovery rate: a practical and powerful approach to multiple testing. J. Roy. Stat. Soc. B 57, 289-300.
83. Rensen E, Pietropaoli S, Mueller F, et al. Sensitive visualization of SARS-CoV-2 RNA with CoronaFISH. Life Sci Alliance. 2022; 5 (4). Epub 2022 Jan. 9.
84. Blair L J, Nordhues B A, Hill S E, et al. Accelerated neurodegeneration through chaperone-mediated oligomerization of tau. J Clin Invest. 2013; 123 (10): 4158-69.
85. Fu H, Possenti A, Freer R, et al. A tau homeostasis signature is linked with the cellular and regional vulnerability of excitatory neurons to tau pathology. Nat Neurosci. 2019; 22 (1): 47-56.
86. Hahle A, Merz S, Meyners C, et al. The Many Faces of FKBP51. Biomolecules. 2019; 9 (1). Epub 2019 Jan. 24.
87. Bosso M, Kirchhoff F. Emerging Role of PYHIN Proteins as Antiviral Restriction Factors. Viruses. 2020; 12 (12). Epub 2020 Dec. 24.
88. Velez J I, Lopera F, Sepulveda-Falla D, et al. APOE*E2 allele delays age of onset in PSEN1 E280A Alzheimer's disease. Mol Psychiatry. 2016; 21 (7): 916-24. Epub 2015 Dec. 2.
89. Yoshiyama Y, Higuchi M, Zhang B, et al. Synapse loss and microglial activation precede tangles in a P301S tauopathy mouse model. Neuron. 2007; 53 (3): 337-51.
90. Jankowsky J L, Zheng H. Practical considerations for choosing a mouse model of Alzheimer's disease. Mol Neurodegener. 2017; 12 (1): 89. Epub 20171222.
91. Mayilsamy K, Markoutsa E, Das M, et al. Treatment with shCCL20-CCR6 nanodendriplexes and human mesenchymal stem cell therapy improves pathology in mice with repeated traumatic brain injury. Nanomedicine: nanotechnology, biology, and medicine. 2020; 29:102247. Epub 2020 Jul. 1.

The disclosures of all publications cited above are expressly incorporated herein by reference, each in its entirety, to the same extent as if each were incorporated by reference individually.

It is also to be understood that the following claims are intended to cover all of the generic and specific features of the invention herein described, and all statements of the scope of the invention which, as a matter of language, might be said to fall there between. Now that the invention has been described,

What is claimed is:

1. A method of treating a neurodegenerative disease in a patient previously infected with a respiratory virus comprising:
obtaining or having obtained a sample from the patient;
obtaining or having obtained an expression level of at least one gene or one gene product in the sample wherein the at least one gene or the at least one gene product is selected from the group consisting of CCL20, CTCFL, CXCL8, EGFR, GFAP, IFI-16, IL-17, IL-18, IL-6R, KLF4, LGALS3, TAC1, CAV1, FKBP5, HSP90, HSPA8, IFITM3, C3/4, C5AR1, CR1, CALB1, CAMKK2, BDNF, CCK/BR, PLAT, MAPT, APP, STAT3, Calbindin, NLRP3, and combinations thereof;
comparing the expression level of the at least one gene or gene expression product to a predetermined control expression level; and
administering a therapeutic agent capable of modulating expression of the at least one gene or the at least one gene product if the expression level of the at least one gene or the at least one gene product is increased as compared to the control level.

2. The method of claim 1 wherein the respiratory virus is severe acute respiratory syndrome coronavirus 2 (SARS CoV-2).

3. The method of claim 2, further comprising diagnosing or having diagnosed the patient with long covid.

4. The method of claim 1, wherein the at least one gene or at least one gene product is selected from the group consisting of FKBP5, IFITM3, IFI16, CR1, C5AR1, and NLRP3.

5. The method of claim 4, wherein at least one of FKBP51, NLRP3, or IFI16 are upregulated as compared to a control.

6. The method of claim 1, wherein the therapeutic agent is an RNA interference agent targeting the at least one gene or gene expression product selected from FKBP51, NLRP3 or IFI16.

7. The method of claim 6, wherein the RNA interference agent is encapsulated within or complexed to an outer surface of a nanoparticle.

8. The method of claim 7, wherein the nanoparticle is a dendrimer nanoformulation wherein the RNA interference agent is complexed to the outer surface of the dendrimer nanoformulation.

9. The method of claim 8, wherein the therapeutic agent is administered intranasally.

10. The method of claim 1, wherein the neurodegenerative disease is selected from the group consisting of Alzheimer's disease (AD), Parkinson's disease (PD), corticobasal degeneration (CBD), progressive supranuclear palsy (PSP), sporadic frontotemporal dementia (FTD), frontotemporal lobar degeneration (FTLD), Lytico-bodig disease (Guam Parkinson-dementia complex), HIV-associated Dementia, multiple sclerosis, amyotrophic lateral sclerosis (ALS), dementias, encephalopathy, and Pick's Disease.

11. The method of claim 10, wherein the neurodegenerative disease is Alzheimer's disease.

12. The method of claim 1, wherein the patient is an aged patient.

13. A method of preventing a neurodegenerative disease in a patient previously infected with a respiratory virus comprising:
obtaining or having obtained a sample from the patient;
obtaining or having obtained an expression level of at least one gene or one gene product in the sample wherein the at least one gene or the at least one gene product is selected from the group consisting of CCL20, CTCFL, CXCL8, EGFR, GFAP, IFI-16, IL-17, IL-18, IL-6R, KLF4, LGALS3, TAC1, CAV1, FKBP5, HSP90, HSPA8, IFITM3, C3/4, C5AR1, CR1, CALB1, CAMKK2, BDNF, CCK/BR, PLAT, MAPT, APP, STAT3, Calbindin, NLRP3, and combinations thereof;
comparing the expression level of the at least one gene or gene expression product to a predetermined control expression level; and
administering a therapeutic agent capable of modulating expression of the at least one gene or the at least one gene product if the expression level of the at least one gene or the at least one gene product is increased as compared to the control level.

14. The method of claim 13, wherein the respiratory virus is severe acute respiratory syndrome coronavirus 2 (SARS CoV-2).

15. The method of claim 14, further comprising diagnosing or having diagnosed the patient with long covid.

16. The method of claim 13, wherein the at least one gene or at least one gene product is selected from the group consisting of FKBP5, IFITM3, IFI16, CR1, C5AR1, and NLRP3.

17. The method of claim 16, wherein at least one of FKBP51, NLRP3, or IFI16 are upregulated as compared to a control.

18. The method of claim 13, wherein the therapeutic agent is an RNA interference agent targeting the at least one gene or gene expression product selected from FKBP51, NLRP3, or IFI16.

19. The method of claim 18, wherein the RNA interference agent is encapsulated within or complexed to an outer surface of a nanoparticle.

20. The method of claim 19, wherein the nanoparticle is a dendrimer nanoformulation wherein the RNA interference agent is complexed to the outer surface of the dendrimer nanoformulation.

21. The method of claim 20, wherein the therapeutic agent is administered intranasally.

22. The method of claim 13, wherein the neurodegenerative disease is Alzheimer's disease or dementia.

23. The method of claim 13, wherein the patient is an aged patient.

24. A method of reducing tauopathy in a patient in need thereof comprising:
obtaining or having obtained a sample from the patient;
obtaining or having obtained an expression level of at least one gene or one gene product in the sample wherein the at least one gene or the at least one gene product is selected from the group consisting of CCL20, CTCFL, CXCL8, EGFR, GFAP, IFI-16, IL-17, IL-18, IL-6R, KLF4, LGALS3, TAC1, CAV1, FKBP5, HSP90, HSPA8, IFITM3, C3/4, C5AR1, CR1, CALB1, CAMKK2, BDNF, CCK/BR, PLAT, MAPT, APP, STAT3, Calbindin, NLRP3, and combinations thereof;
comparing the expression level of the at least one gene or gene expression product to a predetermined control expression level; and
administering a therapeutic agent capable of modulating expression of the at least one gene or the at least one gene product if the expression level of the at least one gene or the at least one gene product is increased as compared to the control level.

25. The method of claim 24, wherein the respiratory virus is severe acute respiratory syndrome coronavirus 2 (SARS CoV-2).

26. The method of claim 25, further comprising diagnosing or having diagnosed the patient with long covid.

27. The method of claim 24, wherein the at least one gene or at least one gene product is selected from the group consisting of FKBP5, IFITM3, IFI16, CR1, C5AR1, and NLRP3.

28. The method of claim 27, wherein at least one of FKBP51, NLRP3, or IFI16 are upregulated as compared to a control.

29. The method of claim 24, wherein the therapeutic agent is an RNA interference agent targeting the at least one gene or gene expression product selected from FKBP51, NLRP3, or IFI16.

30. The method of claim 29, wherein the RNA interference agent is encapsulated within or complexed to an outer surface of a nanoparticle.

31. The method of claim 30, wherein the nanoparticle is a dendrimer nanoformulation wherein the RNA interference agent is complexed to the outer surface of the dendrimer nanoformulation.

32. The method of claim 31, wherein the therapeutic agent is administered intranasally.

33. The method of claim 24, wherein the neurodegenerative disease is Alzheimer's disease or dementia.

34. The method of claim 24, wherein the patient is an aged patient.
